(19) United States
(12) Patent Application Publication
(10) Pub. No.: US 2019/0011463 A1
(43) Pub. Date: Jan. 10, 2019

(54) DIAGNOSIS OF UNSTABLE ANGINA

(71) Applicant: UPSTREAM MEDICAL TECHNOLOGIES LIMITED, Christchurch (NZ)

(72) Inventors: Christopher Joseph Pemberton, Christchurch (NZ); Arthur Mark Richards, Christchurch (NZ)

(21) Appl. No.: 16/065,981

(22) PCT Filed: Dec. 23, 2016
(86) PCT No.: PCT/NZ2016/050207

§ 371 Date: Jun. 25, 2018

Related U.S. Application Data
(60) Provisional application No. 62/271,928, filed on Dec. 28, 2015.

(51) Int. Cl. G01N 33/74; G01N 33/569; G01N 33/84

(52) U.S. Cl. CPC G01N 33/74; G01N 2800/32; G01N 33/84; G01N 33/56972

(57) ABSTRACT

The present invention relates to the diagnosis of unstable angina in a patient. In addition, the present invention relates to predicting the complication of stroke and/or heart failure in a patient as a consequence of developing unstable angina.

### TECHNICAL FIELD

The present invention provides novel assays, methods and kits to diagnose unstable angina in a patient. In addition, the present invention provides novel assays, methods and kits to predict complication of stroke and/or heart failure in a patient as a consequence of unstable angina.

### BACKGROUND OF THE INVENTION

Suspected acute coronary syndromes (ACS) are frequent among hospital emergency department (ED) presentations and comprise between 5-15% of all attendances [1]. The rapid identification of those with genuine myocardial infarction (MI) has been enhanced via the use of highly sensitive cardiac troponin biomarker assays, [2-5] but biomarker assisted identification of those with non-infarction ischemia (eg. unstable angina pectoris (UAP)) is an area of unmet clinical need. UAP is an important clinical substrate for subsequent cardiovascular events and its clear, early identification could help reduce related cardiovascular morbidity and mortality [6].

Applicants have recently provided the first reports that fragments of the signal peptide (sp) regions of the natriuretic hormones B-type natriuretic peptide (BNPsp), A-type natriuretic peptide (ANPsp) and C-type Natriuretic Peptide (CNPsp) are present in the human circulation [7-9]. Both BNPsp and ANPsp display rapid rises in the circulation during ST-elevation MI. BNPsp also shows prompt and significant elevation within 30 minutes in the setting of dobutamine stress echocardiography [10]. Thus, given the need for markers that can discriminate cardiac ischemia, short of tissue necrosis, from other non-cardiac causes of chest pain, Applicants sought to determine the potential of BNPsp, in combination with other markers such as troponin, NT-proBNP and BNP, to improve the early identification of true cardiac ischemia in a prospective study of patients presenting with chest pain suspicious of ACS. Further, Applicants also assessed the prognostic potential of BNPsp alongside troponin, NT-proBNP and BNP in these patients.

### SUMMARY OF THE INVENTION

The inventions described and claimed herein have many attributes and embodiments including, but not limited to, those set forth or described or referenced in this Summary of the Invention. It is not intended to be all-inclusive and the inventions described and claimed herein are not limited to or by the features or embodiments identified in this Summary of the Invention, which is included for purposes of illustration only and not restriction.

Applicants have identified that B-type natriuretic peptide signal peptide fragment defined by residues 17-26 (BNPsp (17-26)), N-terminal B-type natriuretic peptide (NTpro-BNP) and white blood cell count (WCC) is a useful biomarker panel in the diagnosis of unstable angina.

In one aspect the present invention provides a method for diagnosing unstable angina in a patient, the method comprising the steps of:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,

wherein, a ratio of BNPsp fragment and NT-proBNP to WCC that deviates from a reference ratio obtained from a control subject is diagnostic that the patient has unstable angina.

In another aspect the present invention provides a method for predicting a complication of heart failure and/or stroke in a patient who has previously been diagnosed with unstable angina, the method comprising the steps of:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,

wherein, a ratio of BNPsp fragment and NT-proBNP to WCC that deviates from a reference ratio obtained from a control subject is predictive that the patient will develop a complication of heart failure and/or stroke as a consequence of unstable angina.

In a further aspect the present invention provides a method for diagnosing unstable angina in a patient, the method comprising the steps of:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,

wherein, a ratio of BNPsp fragment and NT-proBNP to WCC that deviates from a reference ratio obtained from a control subject is diagnostic that the patient has unstable angina, and wherein in the event of a positive diagnosis of unstable angina:
(iii) administering an intervention therapy so as to reduce, eliminate, ameliorate or treat unstable angina in the patient.

In yet another aspect the present invention provides a test kit for diagnosing unstable angina in a patient, or for predicting complication of heart failure and/or stroke in a patient as a consequence of unstable angina, the test kit comprising:
(i) reagents specific to measure the levels of a Type-B natriuretic peptide signal peptide (BNPsp) fragment, the level of N-terminal Type-B natriuretic peptide (NT-proBNP) and white blood cell count (WCC) in a biological sample obtained from a patient; and
(ii) instructions for how to perform the diagnosis of unstable angina in the patient or for how to predict a complication of stroke and/or heart failure as a consequence of developing unstable angina in the patient.

Applicants have also identified that B-type natriuretic peptide signal peptide fragment defined by residues 17-26 (BNPsp (17-26)), B-type natriuretic peptide (BNP) and white blood cell count (WCC) is a useful biomarker panel in the diagnosis of unstable angina.

Accordingly, in another aspect the present invention provides a method for diagnosing unstable angina in a patient, the method comprising the steps of:
(i) measuring the level of a Type-B natriuretic peptide signal peptide (BNPsp) fragment, the level of Type-B natriuretic peptide (BNP) and white blood cell count (WCC) in a biological sample obtained from the patient; and
(ii) comparing the levels of the BNPsp fragment, BNP and WCC against reference levels obtained from a control subject,

wherein, a ratio of BNPsp fragment and BNP to WCC that deviates from a reference ratio obtained from a control subject is diagnostic that the patient has unstable angina.

In another aspect the present invention provides a method for predicting a complication of heart failure and/or stroke in a patient who has previously been diagnosed with unstable angina, the method comprising the steps of:
(i) measuring the level of a Type-B natriuretic peptide signal peptide (BNPsp) fragment, the level of Type-B natriuretic peptide (BNP) and white blood cell count (WCC) in a biological sample obtained from the patient; and
(ii) comparing the levels of the BNPsp fragment, BNP and WCC against reference levels obtained from a control subject,

wherein, a ratio of BNPsp fragment and BNP to WCC that deviates from a reference ratio obtained from a control subject is predictive that the patient will develop a complication of heart failure and/or stroke as a consequence of unstable angina.

In a further aspect the present invention provides a method for diagnosing unstable angina in a patient, the method comprising the steps of:
(i) measuring the level of a Type-B natriuretic peptide signal peptide (BNPsp) fragment, the level of Type-B natriuretic peptide (proBNP) and white blood cell count (WCC) in a biological sample obtained from the patient; and
(ii) comparing the levels of the BNPsp fragment, BNP and WCC against reference levels obtained from a control subject,

wherein, a ratio of BNPsp fragment and BNP to WCC that deviates from a reference ratio obtained from a control subject is diagnostic that the patient has unstable angina, and wherein in the event of a positive diagnosis of unstable angina:
(iii) administering an intervention therapy so as to reduce, eliminate, mitigate or treat unstable angina in the patient.

In yet another aspect the present invention provides a test kit for diagnosing unstable angina in a patient, or for predicting complication of heart failure and/or stroke in a patient as a consequence of unstable angina, the test kit comprising:
(i) reagents specific to measure the levels of a Type-B natriuretic peptide signal peptide (BNPsp) fragment, the level Type-B natriuretic peptide (BNP) and white blood cell count (WCC) in a biological sample obtained from a patient; and
(ii) instructions for how to perform the diagnosis of unstable angina in the patient or for how to predict a complication of stroke and/or heart failure as a consequence of developing unstable angina in the patient.

### BRIEF DESCRIPTION OF THE FIGURES

FIG. 1 shows a schematic diagram outlining the processing of human preproBNP resulting in generation of free signal peptide, NT-proBNP and BNP peptides;

FIG. 2 shows a Clustal W version 1.83 JALVIEW multiple sequence alignment of the prepoBNP signal peptide sequences. The default Clustal W parameters were used in this alignment as follows: DNA Gap Open Penalty=15.0; DNA Gap Extension Penalty=6.66; DNA matrix=Identity; Protein Gap Open Penalty=10.0; Protein Gap Extension Penalty=0.2; Protein Matrix=Gonnet; Protein/DNA ENDGAP=−1; Protein/DNA GAPDIST=4. The amino acids were submitted in the Fasta format.

FIG. 3 shows receiver operating characteristic (ROC) curve data for the ability of presentation TnI, hsTnT, NT-proBNP and BNPsp to diagnose acute myocardial infarction (MI) in 505 patients presenting with chest pain. BNPsp had an area under the curve (AUC) of 0.69 (P<0.001), which was ˜0.3 points less than either troponin and did not add to either's ability to diagnose MI.

FIG. 4 shows (A) ROC curves of UARatio (AUC=0.70) and NT-proBNP (AUC=0.62) for the identification of UAP (n=40) in 390 non-MI patients at hospital presentation. The UARatio AUC was significantly better than the NT-proBNP AUC (p<0.05). (B) ROC curves of the ability of UARatio, NT-proBNP and BNPsp to diagnose UAP (n=33) in 328 non-MI patients with no abnormalities on ECG. The AUC for UARatio (0.77) was significantly better (p<0.05) than both NT-proBNP (0.66) and BNPsp (0.63).

### SELECTED DEFINITIONS

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used in this specification, the words “comprises”, “comprising”, and similar words, are not to be interpreted in an exclusive or exhaustive sense. In other words, they are intended to mean “including, but not limited to”.

As used in this specification, the term “BNPsp” means B-type Natriuretic Peptide signal peptide. Examples of BNPsp include the full length BNPsp molecule defined by residues 1-27, as well as fragments thereof. In a particular example, BNPsp means the BNPsp fragment defined by residues 17-26 (i.e. BNPsp (17-26; SEQ ID NO:1)).

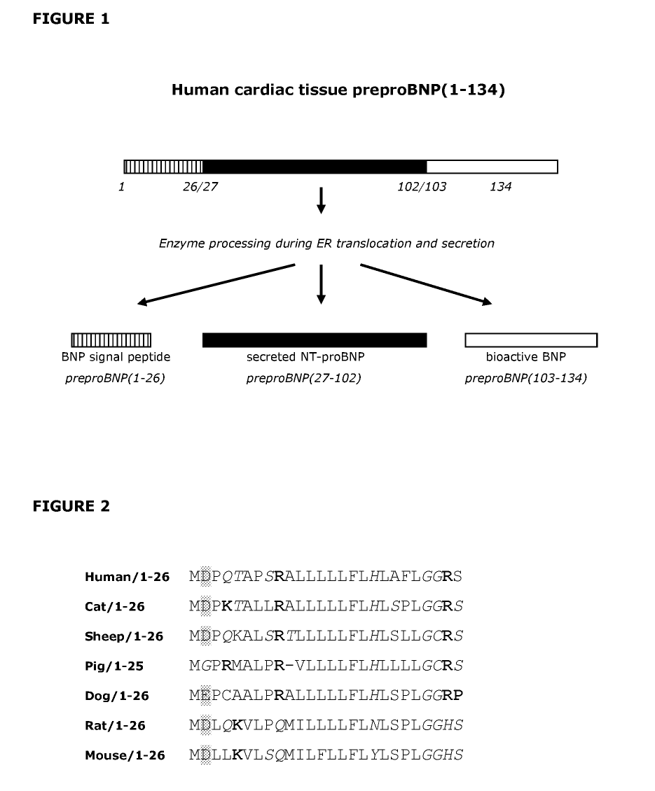

As used in this specification, the term “BNP” means B-type natriuretic peptide, which once processed by proteolytic enzymes includes the N-terminal pro-BNP (NT-proBNP) and the cleaved active form of BNP hormone. For the purpose of this specification, “BNP” refers to BNP(103-134) and “NT-proBNP” refers to NT-proBNP(27-102) as defined in FIG. 1.

As used in this specification, the acronym “STEMI” means ST-elevation myocardial infarction.

As used in this specification, the acronym “NSTEMI” means non ST-elevation myocardial infarction.

As used in this specification, the acronym “UAP” means unstable angina pectoris.

As used in this specification, the acronym “ACS” means acute coronary syndromes.

As used in this specification, the acronym “hsTnT” means highly sensitive troponin T assay.

As used in this specification, the acronym “WCC” means white cell count and relates to the level/number of white blood cells in a sample.

As used in this specification, the acronym “ROC” means receiver operating characteristic curve.

As used in this specification, the acronym “AF” means atrial fibrillation.

As used in this specification, the term “polypeptide” encompasses amino acid chains of any length, including full length sequences in which amino acid residues are linked by covalent peptide bonds. Polypeptides useful in the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof. Polypeptides herein may have chain lengths of at least 4 amino acids, at least 5 amino acids, or at least 6, at least 7, at least 8, at least 9, at least 10, at least 11, at least 12, at least 13, at least 14, at least 15, at least 16, at least 17, at least 18, at least 19, at least 20, at least 21, at least 22, or all 23 amino acids of the full-length EPOsp and/or CNPsp. Reference to other polypeptides of the invention or other polypeptides described herein should be similarly understood.

As used in this specification, the term “fragment” in relation to a polypeptide is a subsequence of a polypeptide that may be detected using a binding agent. The term may refer to a polypeptide, an aggregate of a polypeptide such as a dimer or multimer, a fusion polypeptide, a polypeptide fragment, a polypeptide variant or derivative thereof.

The term “isolated” as applied to the polypeptide sequences disclosed herein is used to refer to sequences that are removed from their natural cellular or other naturally-occurring biological environment. An isolated molecule may be obtained by any method or combination of methods including biochemical, recombinant, and synthetic techniques. The polypeptide sequences may be prepared by at least one purification step.

The term “purified” as used herein does not require absolute purity. Purified refers in various embodiments, for example, to at least about 80%, 85%, 90%, 95%, 98%, or 99% homogeneity of a polypeptide, for example, in a sample. The term should be similarly understood in relation to other molecules and constructs described herein.

As used herein, the term “variant” refers to polypeptide sequences different from the specifically identified sequences, wherein 1 to 6 or more or amino acid residues are deleted, substituted, or added. Substitutions, additions or deletions of one, two, three, four, five or six amino acids are contemplated. Variants may be naturally occurring allelic variants, or non-naturally occurring variants. Variants may be from the same or from other species and may encompass homologues, paralogues and orthologues. In certain embodiments, variants of the polypeptides useful in the invention have biological activities including signal peptide activity or antigenic-binding properties that are the same or similar to those of the parent polypeptides. The term “variant” with reference to polypeptides encompasses all forms of polypeptides as defined herein.

Variant polypeptide sequences exhibit at least about 50%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to a sequence of the present invention. With regard to polypeptides, identity is found over a comparison window of at least 5 to 7 amino acid positions.

Polypeptide variants also encompass those which exhibit a similarity to one or more of the specifically identified sequences that is likely to preserve the functional equivalence of those sequences, including those which could not reasonably be expected to have occurred by random chance. As discussed above, in the case of EPOsp and/or CNPsp variants function may be as either a signal polypeptide, or antigenic polypeptide, or both.

Polypeptide sequence identity and similarity can be determined in the following manner. The subject polypeptide sequence is compared to a candidate polypeptide sequence using BLASTP (from the BLAST suite of programs, version 2.2.18 [April 2008]]) in bl2seq, which is publicly available from NCBI (ftp://ftp.ncbi.nih.gov/blast/). The default parameters of bl2seq are utilized except that filtering of low complexity regions should be turned off.

The similarity of polypeptide sequences may be examined using the following UNIX command line parameters: bl2seq -i peptideseq1 -j peptideseq2 -F F -p blastp. The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match. Variant polypeptide sequences commonly exhibit an E value of less than 1×10⁻⁵, less than 1×10⁻⁶, less than 1×10⁻⁹, less than 1×10⁻¹², less than 1×10⁻¹⁵, less than 1×10⁻¹⁸ or less than 1×10⁻²¹ when compared with any one of the specifically identified sequences. Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity. Use of BLASTP is preferred for use in the determination of polypeptide variants according to the present invention.

The term “binding agent” as used herein refers to any solid or non-solid material capable of binding a species of AMH, fragment or an antigenic variant thereof. In one embodiment the term refers to any natural or non-natural molecule that binds to a species of AMH, fragment or antigenic variant thereof. Examples of binding agents include proteins, peptides, nucleic acids, carbohydrates, lipids, and small molecule compounds. One selective or specific binding agent is an antibody or antigen binding fragment thereof.

The term “antibody” refers to an immunoglobulin molecule capable of specifically binding an antigen, such as, for example, BNPsp, and typically by binding an epitope or antigenic determinant of BNPsp, such as, for example, a C-terminal or N-terminal region of BNPsp. As used herein, the term “antibody” broadly includes full length antibodies and antigen binding fragments or regions thereof. Also included are monoclonal and polyclonal antibodies, multivalent and monovalent antibodies, multispecific antibodies (for example bi-specific antibodies), chimeric antibodies, human antibodies, humanized antibodies and antibodies that have been affinity matured. An antibody binds selectively or specifically to BNPsp, if the antibody binds preferentially to a region or domain of BNPsp which has, e.g. has less than 25%, or less than 10%, or less than 1% or less than 0.1% cross-reactivity with non-BNPsp antigens/epitopes or other non-target BNPsp species, when appropriate. Usually, the antibody will have a binding affinity (dissociation constant (Kd) value), for the antigen or epitope of about 10⁻⁶, or 10⁻⁷M, 10⁻⁸M, or 10⁻⁹M, or 10⁻¹⁰, or 10⁻¹¹ or 10⁻¹²M. Binding affinity may be assessed using surface plasma resonance, for example, or Scatchard analysis.

As used herein, an “antigen binding fragment” or “antibody fragment” or “binding fragment” when used in reference to an antibody, means a portion of the intact antibody that preferably retains most or all, or minimally at least one of, the normal binding functions of the intact antibody. Examples of antibody fragments include Fab, Fab′, F(ab′)₂ and Fv fragments, linear antibodies, diabodies, single chain antibodies (ScFV), domain antibodies and multispecific antibodies.

The term “epitope” includes any antigenic (e.g., a protein) determinant capable of specific binding to an antibody and/or a T cell receptor. That is, a site on an antigen to which B and/or T cells respond. Epitopic determinants usually consist of chemically active surface groupings of molecules such as amino acids or sugar side chains, and usually have specific three-dimensional structural characteristics, as well as specific charge characteristics. An epitope typically includes at least 3, 5 or 8-10 amino acids. The amino acids may be contiguous, or non-contiguous amino acids juxtaposed by tertiary folding. Conformational and non-conformational epitopes are distinguished in that the binding to the former but not the latter is lost in the presence of denaturing solvents.

As used herein, the term “antigenic variant” refers to polypeptide sequences different from the specifically identified sequences, wherein one or more amino acid residues are deleted, substituted, or added. Substitutions, additions or deletions of 1, 2, 3, 4, 5, 6, 10, 15, 20 or more amino acids are specifically contemplated. Variants may be naturally-occurring allelic antigenic variants, or non-naturally occurring antigenic variants. Variants may be from the same or from other species and may encompass homologues, paralogues and orthologues. In certain examples, antigenic variants of the polypeptides useful in the invention have biological activities including hormone function or antigenic-binding properties that are the same or similar to those of the parent polypeptides. The term “antigenic variant” with reference to (poly)peptides encompasses all forms of polypeptides as defined herein. The term “antigenic variant” encompasses naturally occurring, recombinantly and synthetically produced polypeptides. For example, an antigenic variant of human BNP and BNPsp may include the non-human sequences of BNP and BNPsp, such as those sequences derived from mouse, rat, sheep, bovine, pig etc.

In addition to computer/database methods known in the art, polypeptide antigenic variants may be identified by physical methods known in the art, for example, by screening expression libraries using antibodies raised against polypeptides of the invention (Sambrook et al., Molecular Cloning: A Laboratory Manual, 2nd Ed. Cold Spring Harbor Press, 1987) by recombinant DNA techniques also described by Sambrook et al. or by identifying polypeptides from natural sources with the aid of such antibodies.

An “isolated antibody” is an identified antibody that has been separated or recovered, or both, from a component of its natural environment. For example, separated from proteins including enzymes and hormones. In one example, the antibody is purified to at least 95%, or 96% or 97% or 98% or 99% by weight of antibody. Purity can be determined by the Lowry method, for example. Ordinarily the antibody will be prepared by at least one purification step.

As used herein, a “monoclonal antibody” means an antibody that is a highly specific antibody directed against (or which binds to) a single antigen target. A monoclonal antibody may be obtained from a population of homogenous or substantially homogenous antibodies wherein each monoclonal antibody is identical and/or bind the same epitope, except for natural mutations that may occur in minor amounts. Monoclonal antibodies are prepared using methods known the art, such as, for example, in Harlow and Lane (1988) Antibodies, A Laboratory Manual, Cold Spring Harbor Publications, New York, and Harlow and Lane (1999) Using Antibodies: A Laboratory Manual Cold Spring Harbor Laboratory Press, Cold Spring Harbor, N.Y. (jointly and individually referred to herein as Harlow and Lane).

As used herein, a “polyclonal antibody” means an antibody which may be directed against (or which may bind to) multiple antigen targets. Polyclonal antibodies are prepared using methods known the art (such as, for example, in Harlow and Lane, ibid).

The term “ELISA” as used herein means an enzyme linked immunosorbent assay, a type of competitive binding assay comprising antibodies and a detectable label used to quantitate the amount of an analyte in a sample.

The term “capture antibody” as used herein means an antibody which is typically immobilized on a solid support such as a plate, bead or tube, and which antibody binds to and captures analyte(s) of interest, for example membrane bound markers associated with an embryonic stem cell population.

The term “detection antibody” as used herein means an antibody comprising a detectable label that binds to analyte(s) of interest. The label may be detected using routine detection means for a quantitative, semi-quantitative or qualitative measure of the analyte(s) of interest, for example membrane bound markers associated with an embryonic stem cell population.

As used herein, the term “aptamer” refers to single-stranded nucleic acid molecules with secondary structures that facilitate high-affinity binding to a target molecule such as a polypeptide or protein. In certain examples, the single-stranded nucleic acid is ssDNA, RNA or derivatives thereof to improve bioavailability. Aptamer binding affinity to the target protein is further described below.

As used herein, the term “marker” or “biomarker” in the context of an analyte means any antigen, molecule or other chemical or biological entity that is specifically found in circulation or associated with a particular tissue (e.g. heart muscle) that it is desired to be identified in or on a particular tissue affected by a disease or disorder, for example unstable angina. In specific examples, the marker is a circulating peptide (e.g.) BNPsp (17-26) or NT-proBNP. In other examples, the marker is a cell surface antigen or a nuclear antigen that is differentially or preferentially expressed by specific cell types. In other examples the marker is an intracellular antigen that is differentially or preferentially expressed by specific cell types.

The term “ROC” means Receiver Operating Characteristic and a ROC plot depicts the overlap between two distributions by plotting the sensitivity versus 1−specificity for a complete range of decision thresholds.

As used herein, the term “effective amount” refers to the amount of a therapy that is sufficient to result in the prevention of the development, recurrence, or onset of a disease or condition and one or more symptoms thereof, to enhance or improve the prophylactic effect(s) of another therapy, reduce the severity, the duration of disease, ameliorate one or more symptoms of the disease or condition, prevent the advancement of the disease or condition, cause regression of the disease or condition, and/or enhance or improve the therapeutic effect(s) of another therapy.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of the disease or condition. In certain examples, a subject is administered one or more therapies (e.g., one or more prophylactic or therapeutic agents) to “manage” the disease or condition so as to prevent the progression or worsening of the disease or condition.

As used herein, the terms “prevent”, “preventing” and “prevention” in the context of the administration of a therapy to a subject refers to the prevention or inhibition of the recurrence, onset, and/or development of a disease or condition or a symptom thereof in a subject resulting from the administration of a therapy (e.g., a prophylactic or therapeutic agent), or a combination of therapies (e.g., a combination of prophylactic or therapeutic agents).

As used herein, the term “prophylactic agent” refers to any molecule, compound, and/or substance that is used for the purpose of treating unstable angina. Examples of prophylactic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, proliferation based therapy, and small molecule drugs.

As used herein, the term “therapeutic agent” refers to any molecule, compound, and/or substance that is used for the purpose of treating and/or managing a disease or disorder, such as unstable angina. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, biologies, proliferation-based therapy agents, hormonal agents, radioimmunotherapies, targeted agents, epigenetic therapies, differentiation therapies, biological agents, and small molecule drugs.

As used herein, the terms “therapies” and “therapy” can refer to any method(s), composition(s), and/or agent(s) that can be used in the prevention, treatment and/or management of a disease or condition or one or more symptoms thereof.

As used herein, the terms “treat”, “treatment” and “treating” in the context of the administration of a therapy to a subject refer to the reduction, inhibition, elimination or amelioration of the progression and/or duration of (e.g.) unstable angina, the reduction, inhibition, elimination or amelioration of the severity of (e.g.) unstable angina, and/or the amelioration of one or more symptoms thereof resulting from the administration of one or more therapies.

The term “sample” or “biological sample” as used herein means any sample taken or derived from a subject or patient. In this specification, the terms “subject” and “patient” are used interchangeably. Such a sample may be obtained from a subject, or may be obtained from biological materials intended to be provided to the subject. For example, a sample may be obtained from blood or heart tissue being assessed, for example, to investigate the cardiac status in a subject. Included are samples taken or derived from any subjects such as from normal healthy subjects and/or healthy subjects for whom it is useful to understand their cardiac status. Preferred samples are biological fluid samples. The term “biological fluid sample” as used herein refers to a sample of bodily fluid obtained for the purpose of, for example, diagnosis, prognosis, classification or evaluation of a subject of interest, such as a patient. The sample may be any sample known in the art in which peptide antigens may be detected. Included are any body fluids such as a whole blood sample, plasma, serum, ovarian follicular fluid sample, seminal fluid sample, cerebrospinal fluid, saliva, sputum, urine, pleural effusions, interstitial fluid, synovial fluid, lymph, tears, for example, although whole blood sample, plasma and serum are particularly suited for use in this invention. In addition, one of skill in the art would realise that certain body fluid samples would be more readily analysed following a fractionation or purification procedure, for example, separation of whole blood into serum or plasma components.

The term “purified” as used herein does not require absolute purity. Purified refers in one example to at least 90%, or 95%, or 98%, or 99% homogeneity of (e.g.) a polypeptide or antibody in a sample.

The term “subject” and “patient” are used interchangeably herein. These terms preferably refer to a mammal and includes human, and non-human mammals such as cats, dogs, horses, cows, sheep, deer, mice, rats, primates (including gorillas, rhesus monkeys and chimpanzees), possums and other domestic farm or zoo animals. Thus, the assays, methods and kits described herein have application to both human and non-human animals, in particular, and without limitation, humans, primates, farm animals including cattle, sheep, goats, pigs, deer, alpacas, llamas, buffalo, companion and/or pure bred animals including cats, dogs and horses. Preferred subjects are humans, and most preferably “patients” who as used herein refer to living humans who may receive or are receiving medical care or assessment for a disease or condition. Further, while a subject is preferably a living organism, the invention described herein may be used in post-mortem analysis as well.

As used herein, the term “relating to the presence or amount” of an analyte reflects that assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Typically, an analyte is measured in a sample.

A level “higher” or “lower” than a control, or a “change” or “deviation” from a control (level) in one embodiment is statistically significant. A higher level, lower level, deviation from, or change from a control level or mean or historical control level can be considered to exist if the level differs from the control level by about 5% or more, by about 10% or more, by about 20% or more, or by about 50% or more compared to the control level. Statistically significant may alternatively be calculated as P≤0.05. Higher levels, lower levels, deviation, and changes can also be determined by recourse to assay reference limits or reference intervals. These can be calculated from intuitive assessment or non-parametric methods. Overall, these methods may calculate the 0.025, and 0.975 fractiles as 0.025* (n+1) and 0.975 (n+1). Such methods are well known in the art. Presence of a marker absent in a control may be seen as a higher level, deviation or change. Absence of a marker present in a control may be seen as a lower level, deviation or change.

### DETAILED DESCRIPTION

Applicants assessed the ability of B-type natriuretic peptide signal peptide (BNPsp) to assist with the identification of patients with myocardial infarction (MI) and unstable angina pectoris (UAP).

Applicants studied 505 patients who presented to hospital within 4 hours of onset of chest pain suspicious of ACS. Blood samples were drawn at 0, 1, 2 and 24 hours from presentation and assayed for BNPsp, NT-proBNP, TnI and high sensitivity TnT. The ability of BNPsp and other markers to diagnose acute myocardial infarction (MI) and unstable angina pectoris (UAP) and predict subsequent events within one year was then assessed.

Applicants surprisingly discovered that when BNPsp was measured in conjunction with NT-proCNP and white blood cell count, and the data fitted using Receiver Operating Curve analysis, that unstable angina could be diagnosed in a patient presenting to the Emergency Department with symptoms of an acute coronary disorder. Further, the specificity of diagnosis could be enhanced when the levels of potassium (K) in the patient were added to the biomarker panel.

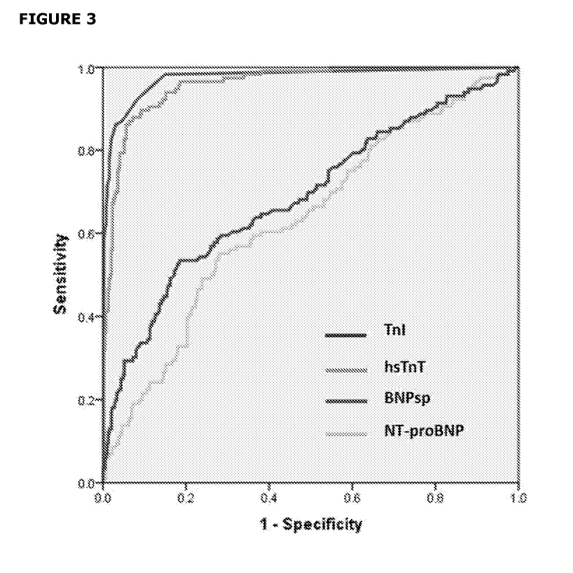

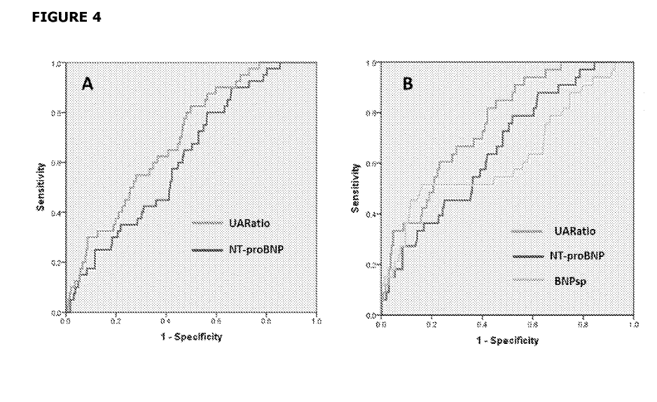

Interestingly, receiver operator area under the curve (AUC) data for the discrimination of myocardial infarction was 0.69 for BNPsp and 0.97 for troponin, with BNPsp failing to add to troponin. However, and importantly, in non-MI patients, BNPsp had discriminative power for UAP (p<0.05), and when combined with presentation values of NT-proBNP, white cell count and potassium into a unique parameter (UARatio), and generated an AUC of 0.76 for UAP in patients with normal ECG results (p<0.001). Refer to FIGS. 3 and 4, as well as Example 2.

Accordingly, in one aspect the present invention provides a method for diagnosing unstable angina in a patient, the method comprising the steps of:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,

wherein, a ratio of BNPsp fragment and NT-proBNP to WCC that deviates from a reference ratio obtained from a control subject is diagnostic that the patient has unstable angina.

Depending on the diagnosis, an intervention therapy may be administered to the patient to reduce, eliminate, mitigate or treat the unstable angina.

Accordingly, in a further aspect the present invention provides a method for diagnosing unstable angina in a patient, the method comprising the steps of:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,

wherein, a ratio of BNPsp fragment and NT-proBNP to WCC that deviates from a reference ratio obtained from a control subject is diagnostic that the patient has unstable angina, and wherein in the event of a positive diagnosis of unstable angina:
(iii) administering an intervention therapy so as to reduce, eliminate, mitigate or treat unstable angina in the patient.

Further, in non-MI patients, the UARatio was significantly predictive of subsequent stroke (AUC=0.70, p<0.05) and heart failure (AUC=0.82, p<0.01) within one year. Refer to Example 2.

Accordingly, in a further aspect the present invention provides a method for predicting a complication of heart failure and/or stroke in a patient who has previously been diagnosed with unstable angina, the method comprising the steps of:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,

wherein, a ratio of BNPsp fragment and NT-proBNP to WCC that deviates from a reference ratio obtained from a control subject is predictive that the patient will develop a complication of heart failure and/or stroke as a consequence of unstable angina.

Depending on the specificity and sensitivity, the diagnosis may be enhanced by measuring the levels of potassium in the sample. As such, in certain examples according to the present invention, the method further comprises measuring the level of potassium in the biological sample obtained from the patient.

In other examples of the present invention, the BNPsp fragment is a fragment defined by residues 17-26 of the full-length/in-tact protein (designated BNPsp (17-26)).

In other examples, the levels of BNPsp, including BNPsp (17-26), and NT-proBNP may be measured by immunoassay or mass spectroscopy. Further details with respect to measurement by immunoassay using antibody- and aptamer-based approaches to detection are given below.

The present invention also contemplates commercial kits and articles of manufacture specific for measuring the levels of, for example, BNPsp fragments, NT-proBNP and white blood cells in a biological sample obtained from a patient. As such, in a further aspect of the present invention there is provided a kit or article of manufacture comprising:
(i) reagents specific to measure the levels of a Type-B natriuretic peptide signal peptide (BNPsp) fragment, the level of N-terminal Type-B natriuretic peptide (NT-proBNP) and white blood cell count (WCC) in a biological sample obtained from a patient; and
(ii) instructions for how to perform the diagnosis of unstable angina in the patient or for how to predict a complication of stroke and/or heart failure as a consequence of developing unstable angina in the patient.

Applicants have also identified that B-type natriuretic peptide signal peptide fragment defined by residues 17-26 (BNPsp (17-26)), B-type natriuretic peptide (BNP) and white blood cell count (WCC) is a useful biomarker panel in the diagnosis of unstable angina.

Accordingly, in another aspect the present invention provides a method for diagnosing unstable angina in a patient, the method comprising the steps of:
(i) measuring the level of a Type-B natriuretic peptide signal peptide (BNPsp) fragment, the level of Type-B natriuretic peptide (BNP) and white blood cell count (WCC) in a biological sample obtained from the patient; and
(ii) comparing the levels of the BNPsp fragment, BNP and WCC against reference levels obtained from a control subject,

wherein, a ratio of BNPsp fragment and BNP to WCC that deviates from a reference ratio obtained from a control subject is diagnostic that the patient has unstable angina.

In another aspect the present invention provides a method for predicting a complication of heart failure and/or stroke in a patient who has previously been diagnosed with unstable angina, the method comprising the steps of:
(i) measuring the level of a Type-B natriuretic peptide signal peptide (BNPsp) fragment, the level of Type-B natriuretic peptide (BNP) and white blood cell count (WCC) in a biological sample obtained from the patient; and
(ii) comparing the levels of the BNPsp fragment, BNP and WCC against reference levels obtained from a control subject,

wherein, a ratio of BNPsp fragment and BNP to WCC that deviates from a reference ratio obtained from a control subject is predictive that the patient will develop a complication of heart failure and/or stroke as a consequence of unstable angina.

In a further aspect the present invention provides a method for diagnosing unstable angina in a patient, the method comprising the steps of:
(i) measuring the level of a Type-B natriuretic peptide signal peptide (BNPsp) fragment, the level of Type-B natriuretic peptide (proBNP) and white blood cell count (WCC) in a biological sample obtained from the patient; and
(ii) comparing the levels of the BNPsp fragment, BNP and WCC against reference levels obtained from a control subject,

wherein, a ratio of BNPsp fragment and BNP to WCC that deviates from a reference ratio obtained from a control subject is diagnostic that the patient has unstable angina, and wherein in the event of a positive diagnosis of unstable angina:
(iii) administering an intervention therapy so as to reduce, eliminate, mitigate or treat unstable angina in the patient.

In yet another aspect the present invention provides a test kit for diagnosing unstable angina in a patient, or for predicting complication of heart failure and/or stroke in a patient as a consequence of unstable angina, the test kit comprising:
(i) reagents specific to measure the levels of a Type-B natriuretic peptide signal peptide (BNPsp) fragment, the level Type-B natriuretic peptide (BNP) and white blood cell count (WCC) in a biological sample obtained from a patient; and
(ii) instructions for how to perform the diagnosis of unstable angina in the patient or for how to predict a complication of stroke and/or heart failure as a consequence of developing unstable angina in the patient.

### Antibodies and Antigen Binding Fragments

As noted above, antibody or antibodies as used herein refers to a peptide or polypeptide derived from, modelled after or substantially encoded by an immunoglobulin gene or immunoglobulin genes, or fragments thereof, capable of specifically binding an antigen or epitope [34-36]. As foreshadowed in the definition section of this specification, the term antibody includes antigen binding fragments such as, for example, fragments, subsequences, complementarity determining regions (CDRs) that retain capacity to bind to an antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment [37], which consists of a VH domain; and (vi) an isolated complementarity determining region (CDR). Single chain antibodies are also included by reference in the term “antibody.” Further discussion of antibodies and fragments may be found in references (e.g.) [38-44] all of which are incorporated herein in their entirety.

Also included is antiserum obtained by immunizing an animal such as a mouse, rat or rabbit with an antigen, such as for example, BNPsp or BNPsp fragments, as well as antigenic variants thereof. In brief, methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include BNPsp or BNPsp fragments, antigenic variants thereof or a fusion protein thereof. It may be useful to conjugate the immunizing agent to a protein known to be immunogenic in the mammal being immunized. Examples of such immunogenic proteins include but are not limited to keyhole limpet hemocyanin, bovine serum albumin, bovine thyroglobulin, and soybean trypsin inhibitor. Examples of adjuvants that may be employed include Freund's complete adjuvant and MPL TDM adjuvant (monophosphoryl Lipid A, synthetic trehalose dicorynomycolate). The immunization protocol may be selected by one skilled in the art without undue experimentation.

Monoclonal antibodies may be prepared using hybridoma methods well known in the art [e.g. 45-47]. The hybridoma cells may be cultured in a suitable culture medium, alternatively, the hybridoma cells may be grown in vivo as ascites in a mammal. Preferred immortalized cell lines are murine myeloma lines, which can be obtained, for example, from the American Type Culture Collection, Virginia, USA. Immunoassays may be used to screen for immortalized cell lines that secrete the antibody of interest. Sequences of BNPsp or BNPsp fragments or antigenic variants thereof may be used in screening.

Well known means for establishing binding specificity of monoclonal antibodies produced by the hybridoma cells include immunoprecipitation, radiolinked immunoassay (RIA), enzyme-linked immunoabsorbent assay (ELISA) and Western blot [48]. For example, as noted above, the binding affinity of the monoclonal antibody can, for example, be determined by the Scatchard analysis [49]. Samples from immunised animals may similarly be screened for the presence of polyclonal antibodies.

Monoclonal antibodies can also be obtained from recombinant host cells. DNA encoding the antibody can be obtained from a hybridoma cell line. The DNA is then placed into an expression vector, transfected into host cells (e.g., COS cells, CHO cells, E. coli cells) and the antibody produced in the host cells. The antibody may then be isolated and/or purified using standard techniques.

The monoclonal antibodies or fragments may also be produced by recombinant DNA means (e.g. [50]). DNA modifications such as substituting the coding sequence for human heavy and light chain constant domains in place of the homologous murine sequences [50] are also possible. The antibodies may be monovalent antibodies. Methods for preparing monovalent antibodies are well known in the art (e.g. [51-53]. Production of chimeric [54], bivalent antibodies [55] and multivalent antibodies are also contemplated herein [56].

Other known art techniques for monoclonal antibody production such as from phage libraries, may also be used (e.g. [57]).

The monoclonal antibodies secreted by the cells may be isolated or purified from the culture medium or ascites fluid by conventional immunoglobulin purification procedures such as, for example, reverse phase HPLC, protein A-Sepharose, hydroxyapatite chromatography, gel electrophoresis, dialysis, or affinity chromatography [58].

Bispecific antibodies may also be useful. These antibodies are monoclonal, preferably human or humanized, antibodies that have binding specificities for at least two different antigens. Antibodies with greater than two specificities for example trispecific antibodies are also contemplated herein.

Antibodies used in the immunoassays described herein specifically bind to BNPsp or BNPsp fragments. The term “specifically binds” is not intended to indicate that an antibody binds exclusively to its intended target since, as noted above, an antibody binds to any polypeptide displaying the epitope(s) to which the antibody binds. Rather, an antibody “specifically binds” if its affinity for its intended target is about 5-fold greater when compared to its affinity for a non-target molecule which does not display the appropriate epitope(s). In certain examples, the affinity of the antibody will be at least about 5 fold, preferably 10 fold, more preferably 25-fold, even more preferably 50-fold, and most preferably 100-fold or more, greater for a target molecule than its affinity for a non-target molecule. In other examples, antibodies bind with affinities of at least about 10⁻⁶M, or 10⁻⁷M, or at least about 10⁻⁸M, or 10⁻⁹M, or 10⁻¹⁰, or 10⁻¹¹ or 10⁻¹²M.

Affinity is calculated as K_{d}=k_{off}/kₒₙ (k_{off} is the dissociation rate constant, Kₒₙ is the association rate constant and K_{d} is the equilibrium constant). Affinity can be determined at equilibrium by measuring the fraction bound (r) of labelled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art [59].

Numerous publications discuss the use of phage display technology to produce and screen libraries of polypeptides for binding to a selected analyte [60-63]. A basic concept of phage display methods is the establishment of a physical association between DNA encoding a polypeptide to be screened and the polypeptide. This physical association is provided by the phage particle, which displays a polypeptide as part of a capsid enclosing the phage genome that encodes the polypeptide. The establishment of a physical association between polypeptides and their genetic material allows simultaneous mass screening of very large numbers of phage bearing different polypeptides. Phage displaying a polypeptide with affinity to a target binds to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means (e.g. [64]).

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labelled secondary antibody (for example, an anti-mouse antibody conjugated to alkaline phosphatase if the raised antibodies are mouse antibodies) is added to the wells and incubated for about 30 min and then washed. Substrate is added to the wells and a colour reaction will appear where antibody to the immobilized polypeptide(s) is present.

The antibodies so identified may then be further analysed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

### Aptamers

The present invention also contemplates aptamers that selectively bind to analytes of interest including, for example, BNP, BNPsp and fragments thereof.

Nucleic acid aptamers are nucleic acid species that have been engineered through repeated rounds of in vitro selection equivalently, SELEX (systematic evolution of ligands by exponential enrichment) to bind to various molecular targets such as small molecules, proteins, nucleic acids, and even cells, tissues and organisms. Aptamers offer molecular binding and recognition equivalent to antibodies. In addition to their discriminate recognition, aptamers offer advantages over antibodies as they can be engineered completely in vitro, are readily produced by chemical synthesis, possess desirable storage properties, and elicit little or no immunogenicity in therapeutic applications.

According to an example of the present invention, the aptamer is a monomer (one unit). According to another example of the invention, the aptamer is a multimeric aptamer. The multimeric aptamer may comprise a plurality of aptamer units (mers). Each of the plurality of units of the aptamer may be identical. In such a case the multimeric aptamer is a homomultimer having a single specificity but enhanced avidity (multivalent aptamer).

Alternatively, the multimeric aptamer may comprise two or more aptameric monomers, wherein at least two mers of the multimeric aptamer are non-identical in structure, nucleic acid sequence or both. Such a multimeric aptamer is referred to herein as a heteromultimer. The heteromultimer may be directed to a single binding site i.e., monospecific (such as to avoid steric hindrance). The heteromultimer may be directed to a plurality of binding sites i.e., multispecific. The heteromultimer may be directed to a plurality of binding sites on different analytes, including for example, BNP, BNPsp and fragments thereof. Further description of the multimeric aptamer is provided hereinbelow.

A plurality of multimeric aptamers may be conjugated to form a conjugate of multimeric aptamers. The multimeric aptamer may comprise, two (dimer), three (trimer), four (tetramer), five (pentamer), six (hexamer), and even more units.

Aptamers of the invention can be synthesized and screened by any suitable methods in the art.

For example, aptamers can be screened and identified from a random aptamer library by SELEX (systematic evolution of ligands by exponential enrichment). Aptamers that bind to an antigen of interest can be suitably screened and selected by a modified selection method herein referred to as cell-SELEX or cellular-SELEX [30-32]. In other examples, aptamers that bind to a cell surface target molecule (e.g., BNP or BNPsp) can be screened by capillary electrophoresis and enriched by SELEX based on the observation that aptamer-target molecule complexes exhibited retarded migration rate in native polyacrylamide gel electrophoresis as compared to unbound aptamers.

A random aptamer library can be created that contains monomeric, dimeric, trimeric, tetrameric or other higher multimeric aptamers. A random aptamer library (either ssDNA or RNA) can be modified by including oligonucleotide linkers to link individual aptamer monomers to form multimeric aptamer fusion molecules. In other examples, a random oligonucleotide library is synthesized with randomized 45 nt sequences flanked by defined 20 nt sequences both upstream and downstream of the random sequence, i.e., known as 5′-arm and 3′-arm, which are used for the amplification of selected aptamers. A linking oligonucleotide (i.e., linker) is designed to contain sequences complementary to both 5′-arm and 3′-arm regions of random aptamers to form dimeric aptamers. For trimeric or tetrameric aptamers, a small trimeric or tetrameric (i.e., a Holiday junction-like) DNA nanostructure is engineered to include sequences complementary to the 3′-arm region of the random aptamers, therefore creating multimeric aptamer fusion through hybridization. In addition, 3-5 or 5-10 dT rich nucleotides can be engineered into the linker polynucleotides as a single stranded region between the aptamer-binding motifs, which offers flexibility and freedom of multiple aptamers to coordinate and synergize multivalent interactions with cellular ligands or receptors. Alternatively, multimeric aptamers can also be formed by mixing biotinylated aptamers with streptavidin.

A modified cellular SELEX procedure can be employed to select target-binding aptamers. Multimeric aptamers may be multivalent but be of single binding specificity (i.e., homomultimeric aptamers). Alternatively, the multimeric aptamer may be multivalent and multi-specific (i.e., heteromultimeric aptamers).

Thus, each monomer of the homomultimeric aptamer binds the target protein (e.g., BNP, BNPsp or fragments thereof) in an identical manner. Thus according to an example of the invention, all monomeric components of the homomultimeric aptamer are identical.

Conversely, a heteromultimeric aptamer comprises a plurality of monomeric aptamers at least two of which bind different sites on a single target protein or bind at least two different target proteins.

Selection of RNA-aptamers is well-established using protocols described in the scientific literature (e.g. [33]).

In certain examples, a suitable nucleotide length for an aptamer ranges from about 15 to about 100 nucleotide (nt), and in various other examples, 12-30, 14-30, 15-30 nt, 30-100 nt, 30-60 nt, 25-70 nt, 25-60 nt, 40-60 nt, or 40-70 nt in length.

In other examples, the aptamer has affinity at the range of 10-100 nM, which, after binding of the aptamer to a tumor cell surface molecule, permits dissociation of the aptamer from the target molecule (e.g., BNP or BNPsp), which leads to the release and recycle of the aptamer nucleic acid nanostructure to target other tumor cells. The affinity of individual aptamers can be increased by 4-50 fold by constructing multimeric aptamers linked together by covalent or non-covalent linkages. Methods of multimerizing aptamers are further described hereinbelow.

Thus, in certain examples, the desirable affinity of an aptamer to an analyte of interets (e.g. BNP or BNPsp) can be fine-tuned by adjusting the multiplexity of the monomeric aptamer.

Multimerization can be done at the library level as follows.

In certain examples, a linker polynucleotide has a length between about 5 nucleotides (nt) and about 100 nt; in various examples, 10-30 nt, 20-30 nt, 25-35 nt, 30-50 nt, 40-50 nt, 50-60 nt, 55-65 nt, 50-80 nt, or 80-100 nt. It is within the ability of one of skill in the art to adjust the length of the linker polynucleotide to accommodate each monomeric aptamer in the multimeric structure.

In certain examples, the multimeric aptamers can be identified and screened from a random multimeric aptamer library as described herein. In other examples, the monomeric aptamers are linked to each other by one or a plurality of linker polynucleotides to form multimeric aptamers. Monomeric aptamers can be linked to form multimeric aptamers by any suitable means and in any configurations.

It will be appreciated that the monomeric structures of the invention can be further multimerized by post SELEX procedures.

Multimers can be linearly linked by continuous linear synthesis of DNA without spacers or with nucleic acid spacers. Aptamer synthesis usually relies on standard solid phase phosphoramitide chemistry.

Thus, dimers, trimers and tetramers or higher oligomeric structures (e.g., pentamers, hexamers, heptamers, octamers etc.) can be linked by a polymeric spacer. Methods of generating such polymeric structures are provided in (e.g.) [65].

In certain examples, the aptamers are further modified to protect the aptamers from nuclease and other enzymatic activities. The aptamer sequence can be modified by any suitable methods known in the art. For example, phosphorothioate can be incorporated into the backbone, and 5′-modified pyrimidine can be included in 5′ end of ssDNA for DNA aptamer. For RNA aptamers, modified nucleotides such as substitutions of the 2′—OH groups of the ribose backbone, e.g., with 2′-deoxy-NTP or—fluoro-NTP, can be incorporated into the RNA molecule using T7 RNA polymerase mutants. The resistance of these modified aptamers to nuclease can be tested by incubating them with either purified nucleases or nuclease from mouse serum, and the integrity of aptamers can be analyzed by gel electrophoresis.

The monomeric or multimeric aptamer of the invention can be further attached or conjugated to a detectable or therapeutic moiety (i.e., a pharmaceutical moiety).

Thus, as noted above, a diagnostic or therapeutic moiety can be attached to an aptamer embodied herein to provide additional biological activity, such as for diagnosing, preventing, or treating a condition or disease. In one example a diagnostic moiety such as a detectable moiety e.g., label (e.g., His tag, flag tag), fluorescent, radioactive, biotin/avidin etc., can be bound to the aptamer, and imaging, immunohistochemistry, or other invasive or non-invasive methods used to identify the location(s) and extend of binding of the conjugate to locations within the body. For therapeutic uses, a cytotoxic agent such as a chemotherapeutic agent, radioactive moiety, toxin, antibody, nucleic acid silencing agents e.g., small interfering RNA (siRNA) or other molecule with therapeutic activity when delivered to cells expressing a molecule to which the aptamer is targeted, may be used to enhance the therapeutic activity of the aptamer or provide a biological activity where the aptamer is providing the targeting activity. Moreover, other conjugates to the aptamers described herein are contemplated, such as but not limited to scaffolds, sugars, proteins, antibodies, polymers, and nanoparticles, each of which have art-recognized therapeutic or diagnostic utilities and can be targeted to particular sites in vivo using an aptamer embodied herein.

### Detection of Binding Agents

The present disclosure includes use of a detection system involving the binding of analytes of interest, including but not limited to BNP, BNPsp and fragments thereof, to a binding agent and then detecting the amount of bound agent. A similar solution is to detect the amount of unbound binding agent in a sample to get an indication of unbound or bound peptide or protein of interest. It is intended that such alternative methods fall within the scope of the present disclosure as functional alternatives to directly detecting the amount of bound binding agent. Persons skilled in the art will appreciate that the concentration of BNP, BNPsp and fragments thereof in a sample can be readily calculated from the amount of BNP, BNPsp and fragments thereof in a sample when the sample volume is known.

The antibodies according to the present disclosure are particularly useful in immunoassays for determining the presence and/or amount of BNP, BNPsp and fragments thereof in a sample. Due to variable binding affinities of different antibodies, the person skilled in the art will appreciate that a standard binding curve of measured values versus amount of protein in a sample should be established for a particular antibody to enable the amount of protein in a sample to be determined. Such a curve is then used to determine the true amount of protein in a sample. In other words, a reference interval needs to be determined for each binding agent, including antibody, used.

Sample materials include biological fluids but are not limited thereto. In terms of the present disclosure, the biological fluid is typically blood. In one example, the sample is tested in vitro.

Immunoassays specific for BNP, BNPsp and fragments thereof require the production of antibodies that specifically bind to BNP, BNPsp and fragments thereof. Antibodies can be used to construct immunoassays with broad specificity, as in competitive binding assays below, or used in conjunction with other antibodies described below in sandwich type assays to produce assays specific to each of the three peptides or to other peptides of interest. The person skilled in the art will appreciate that non-competitive assays are also possible. Refer below.

The methods of the present disclosure can be performed using a kit as provided herein. A kit for measuring the level of BNP, BNPsp and fragments thereof in a biological sample is provided. The kit comprises a binding agent that selectively binds to BNP, BNPsp and fragments thereof and which can be quantitatively measured upon binding to BNP, BNPsp and fragments thereof. Binding agents are as described above.

In another example, indicators may also be used. Indicators may be employed in ELISA and RIA methods.

Polyclonal and monoclonal antibodies can be used in competitive binding or sandwich or dipstick type assays. In one example of this method a liquid sample is contacted with the antibody and simultaneously or sequentially contacted with a labelled BNP, BNPsp and fragments or modified peptide containing the epitope recognised by the antibody.

The label can be a radioactive component such as ¹²⁵I, ¹³¹I, ³H, ¹⁴C or a nonradioactive component that can be measured by time resolved fluorescence, fluorescence, fluorescence polarisation, luminescence, chemiluminescence or colorimetric methods. These compounds include europium or other actinide elements, acrinidium esters, fluorescein, or radioactive material such as those above, that can be directly measured by radioactive counting, measuring luminescent or fluorescent light output, light absorbance etc. The label can also be any component that can be indirectly measured such as biotin, digoxin, or enzymes such as horseradish peroxidase, alkaline phosphatase. These labels can be indirectly measured in a multitude of ways. Horseradish peroxidase for example can be incubated with substrates such as o-Phenylenediamine Dihyhdrochloride (OPD) and peroxide to generate a coloured product whose absorbance can be measured, or with luminol and peroxide to give chemiluminescent light that can be measured in a luminometer. Biotin or digoxin can be reacted with binding agents that bind strongly to them; e.g. avidin will bind strongly to biotin. These binding agents can in turn be covalently bound or linked to measurable labels such as horseradish peroxidase or other directly or indirectly measured labels as above. These labels and those above may be attached to the peptide or protein: —during synthesis, by direct reaction with the label, or through the use of commonly available crosslinking agents such as MCS and carbodiimide, or by addition of chelating agents.

Following contact with the antibody, usually for 18 to 25 hours at 4° C., or 1 to 240 minutes at 30° C. to 40° C., the labelled peptide bound to the binding agent (for example, antibody) is separated from the unbound labelled peptide. In solution phase assays, the separation may be accomplished by addition of an anti gamma globulin antibody (second-antibody) coupled to solid phase particles such as cellulose, or magnetic material. The second-antibody is raised in a different species to that used for the primary antibody and binds the primary antibody. All primary antibodies are therefore bound to the solid phase via the second antibody. This complex is removed from solution by centrifugation or magnetic attraction and the bound labelled peptide measured using the label bound to it. Other options for separating bound from free label include formation of immune complexes, which precipitate from solution, precipitation of the antibodies by polyethyleneglycol or binding free labelled peptide to charcoal and removal from solution by centrifugation of filtration. The label in the separated bound or free phase is measured by an appropriate method such as those presented above.

Competitive binding assays can also be configured as solid phase assays that are easier to perform and are therefore preferable to those above. This type of assay uses plates with wells (commonly known as ELISA or immunoassay plates), solid beads or the surfaces of tubes. The primary antibody is either adsorbed or covalently bound to the surface of the plate, bead or tube, or is bound indirectly through a second anti-gamma globulin or anti Fc region antibody adsorbed or covalently bound to the plate. Sample and labelled peptide (as above) are added to the plate either together or sequentially and incubated under conditions allowing competition for antibody binding between BNP, BNPsp and fragments thereof in the sample and the labelled peptide. Unbound labelled peptide can subsequently be aspirated off and the plate rinsed leaving the antibody bound labelled peptide attached to the plate. The labelled peptide can then be measured using techniques described above.

Sandwich type assays are more preferred for reasons of specificity, speed and greater measuring range. In this type of assay an excess of the primary antibody to BNP, BNPsp and fragments thereof is attached to the well of an ELISA plate, bead or tube via adsorption, covalent coupling, or an anti Fc or gamma globulin antibody, as described above for solid phase competition binding assays. Sample fluid or extract is contacted with the antibody attached to the solid phase. Because the antibody is in excess this binding reaction is usually rapid. A second antibody to BNP, BNPsp and fragments thereof is also incubated with the sample either simultaneously or sequentially with the primary antibody. This second antibody is chosen to bind to a site on BNP, BNPsp and fragments thereof that is different from the binding site of the primary antibody. These two antibody reactions result in a sandwich with the BNP, BNPsp and fragments thereof from the sample sandwiched between the two antibodies. The second antibody is usually labelled with a readily measurable compound as detailed above for competitive binding assays. Alternatively a labelled third antibody that binds specifically to the second antibody may be contacted with the sample. After washing the unbound material the bound labelled antibody can be measured by methods outlined for competitive binding assays. After washing away the unbound labelled antibody, the bound label can be quantified as outlined for competitive binding assays.

### Immunoassays

In general, immunoassays involve contacting a sample containing or suspected of containing a peptide biomarker of interest with at least one antibody that specifically binds to the biomarker. A signal is then generated indicative of the presence or amount of complexes formed by the binding of peptides in the sample to the antibody. The signal is then related to the presence or amount of the peptide biomarker in the sample (quantitatively, semi-quantitatively or qualitatively). Numerous methods and devices are well known to the skilled artisan for the detection and analysis of peptide biomarkers (e.g. [66-79].

The assay devices and methods according to the present invention may utilize labelled molecules in various sandwich, competitive, or non-competitive assay formats to generate a signal that is related to the presence or amount of, for example, BNP or BNPsp, or fragments thereof in a sample. Suitable assay formats used for the present invention include in particular, enzyme-linked immunoassays (ELISA), radioimmunoassays (RIAs), competitive binding assays, and the like. Also contemplated are chromatographic, mass spectrographic, and protein “blotting” methods. Additionally, certain methods and devices, such as biosensors and optical immunoassays, may be employed to determine the presence or amount of analytes without the need for a labelled molecule [80, 81]. One skilled in the art also recognizes that robotic instrumentation including but not limited to Beckman ACCESS®™, Abbott AXSYM®™, Roche ELECSYS®™, Dade Behring STRATUS®™ systems are among the immunoassay analyzers that are capable of performing immunoassays described here, as an example of the present invention.

Antibodies or other polypeptides may be immobilized onto a variety of solid supports for use in the assays and methods of the present invention. Solid supports or phases that may be used to immobilize specific binding agents include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colour spot. Antibodies or other polypeptides may be bound to specific zones of assay devices either by conjugating directly to an assay device surface, for example, or by indirect binding. In an example of the latter case, antibodies or other polypeptides may be immobilized on particles or other solid supports, and that solid support immobilized to the device surface.

Biological assays require methods for detection, and one of the most common methods for quantitation of results is to conjugate a detectable label to a protein that has affinity for one of the components in the biological system or sample being studied. In the assays and methods of the present invention, the detectable label is typically conjugated to a binding agent, such as an antibody. Binding of BNPsp or fragments thereof to an antibody to form a complex can be detected directly or indirectly. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

By way of illustration, horseradish peroxidase for example can be incubated with substrates such as o-Phenylenediamine Dihyhydrochloride (OPD) and peroxide to generate a coloured product whose absorbance can be measured, or with luminol and peroxide to give chemiluminescent light which can be measured in a luminometer as is known in the art. Biotin or digoxin can be reacted with binding agents that bind strongly to them. For example, the proteins avidin and streptavidin will bind strongly to biotin. A further measurable label is then covalently bound or linked thereto either by direct reaction with the protein, or through the use of commonly available crosslinking agents such as carbodiimide, or by addition of chelating agents.

Detection also includes fluorescence resonance energy transfer (FRET) between fluorescent labels, particularly in dual assay formats according to the present invention for the simultaneous measurement of, for example, BNP, BNPsp and fragments thereof.

As such, the present invention also contemplates the analysis of different species of BNP and BNPsp, such as the detection and measurement of BNPsp (17-26) for example, using multi-site assay formats, as will be known to a person skilled in the art (e.g. [82]).

Generation of a signal from the label can be performed using various optical, acoustical, and electrochemical methods well known in the art. As described herein, examples of detection modes include fluorescence, radiochemical detection, reflectance, absorbance, amperometry, conductance, impedance, interferometry, ellipsometry, etc. This list is not meant to be limiting. Antibody-based biosensors may also be employed to determine the presence or amount of analytes that optionally eliminate the need for a labelled molecule.

Immunoassay analysers are also well known and include Beckman Access, Abbott AxSym, Roche ElecSys and Dade Behring Status systems amongst others that are well described.

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

Sandwich type assays (a type of competitive binding assay) have greater specificity, speed and greater measuring range. In this type of assay an excess of the primary antibody to BNPsp or BNPsp fragment is attached to the well of an ELISA plate, bead or tube via adsorption, covalent coupling, or a second antibody, as described above for solid phase competition binding assays. Sample fluid or extract is contacted with the antibody attached to the solid phase. Because the antibody is in excess this binding reaction is usually rapid. A detection antibody to BNP or BNPsp is also incubated with the sample either simultaneously or sequentially with the primary antibody. The detection antibody is chosen to bind to a site on BNP or BNPsp that is different from the binding site of the primary antibody. These two antibody reactions result in a sandwich with the BNP or BNPsp or fragment from the sample sandwiched between the two antibodies. The detection antibody is usually labelled with a readily measurable compound as detailed above. Alternatively a labelled third antibody that binds specifically to the detection antibody may be contacted with the sample. After washing away the unbound material the bound labelled antibody can be measured and quantified by methods outlined for competitive binding assays.

In certain examples of the present invention, various types of immunoassays are used, which may include a competitive type of immunoassay. Examples of competitive immunoassays include an enzyme immunoassay or enzyme-linked immunosorbent assay (EIA or ELISA), a fluorescent immunoassay, a radiometric or radioimmunoassay (RIA), a magnetic separation assay (MSA), a lateral flow assay, a diffusion immunoassay, an immunoprecipitation assay, an immunosorbent or “antigen-down” assay using an analyte bound to a solid support, or an agglutination assay. In one such assay, a sample contains an unknown amount of analyte to be measured, which may be a protein such as BNPsp or BNPsp fragment. The analyte may also be termed an antigen. The sample may be spiked with a known or fixed amount of labelled analyte. The spiked sample is then incubated with an antibody that binds to the analyte, such as BNP or BNPsp or fragments thereof, so that the analyte in the sample and the labelled analyte added to the sample compete for binding to the available antibody binding sites. More or less of the labelled analyte will be able to bind to the antibody binding sites, depending on the relative concentration of the unlabelled analyte present in the sample. Accordingly, when the amount of labelled analyte bound to the antibody is measured, it is inversely proportional to the amount of unlabelled analyte in the sample. The amount of analyte in the original sample may then be calculated based on the amount of labelled analyte measured, using standard techniques known in the art.

In another type of competitive immunoassay, an antibody that binds to the analyte, such as BNP or BNPsp or fragments thereof, may be coupled with or conjugated to a ligand, wherein the ligand binds to an additional antibody added to the sample. One example of such a ligand includes fluorescein. The additional antibody may be bound to a solid support. The additional antibody binds to the ligand coupled with the antibody that binds in turn to the analyte or alternatively to the labelled analyte, forming a mass complex which allows isolation and measurement of the signal generated by the label coupled with the labelled analyte.

In another type of competitive immunoassay, the analyte to be measured may be bound to a solid support, and incubated with both an antibody that binds to the analyte and a sample containing the analyte to be measured. The antibody binds to either the analyte bound to the solid support or to the analyte in the sample, in relative proportions depending on the concentration of the analyte in the sample. The antibody that binds to the analyte bound to the solid support is then bound to another antibody, such as anti-mouse IgG, that is coupled with a label. The amount of signal generated from the label is then detected to measure the amount of antibody that bound to the analyte bound to the solid support. Such a measurement will be inversely proportional to the amount of analyte present in the sample. Such an assay may be used in a microtiter plate format.

Examples of the present invention as disclosed herein may be used to perform immunoassays referred to as immunometric, “two-site” or “sandwich” immunoassays, wherein the analyte may be bound to or sandwiched between two antibodies that bind to different epitopes on the analyte, such as BNP or BNPsp or fragments thereof. Representative examples of such immunoassays include enzyme immunoassays or enzyme-linked immunosorbent assays (EIA or ELISA), immunoradiometric assays (IRMA), fluorescent immunoassays, lateral flow assays, diffusion immunoassays, immunoprecipitation assays, and magnetic separation assays (MSA). In one such assay, a first antibody, which may be described as the “capture” antibody, may be bound to a solid support, for which examples have been listed above. The capture antibody may be bound to or coated on a solid support using procedures known in the art. Alternatively, the capture antibody may be coupled with a ligand that is recognized by an additional antibody that is bound to or coated on a solid support. Binding of the capture antibody to the additional antibody via the ligand then indirectly immobilizes the capture antibody on the solid support. An example of such a ligand is fluorescein. The second antibody, which may be described as the “detection” antibody, may be coupled with a label, which may comprise a chemiluminescent agent, a calorimetric agent, an energy transfer agent, an enzyme, a fluorescent agent or a radioisotope. The detection antibody may be coupled with or conjugated with a label using procedures known in the art. The label may comprise a first protein such as biotin coupled with the second antibody, and a second protein such as streptavidin that is coupled an enzyme. The second protein binds to the first protein. The enzyme produces a detectable signal when provided with substrate(s), so that the amount of signal measured corresponds to the amount of second antibody that is bound to the analyte. Horseradish peroxidase is an example of such an enzyme; possible substrates include TMB (3,3′,5,5′-tetramethyl benzidine, OPD (o-phenylene diamine), and ABTS (2,2′-azino-bis(3-ethylbenzthiazoline-6-sulfonic acid).

Sandwich immunoassays or sandwich ELISAs are particularly suited for use in the present invention.

A dipstick type assay may also be used. These assays are well known in the art. They may for example, employ small particles such as gold or coloured latex particles with specific antibodies attached. The liquid sample to be measured may be added to one end of a membrane or paper strip preloaded with the particles and allowed to migrate along the strip. Binding of the antigen (such as BNP, BNPsp or fragments thereof) in the sample to the particles modifies the ability of the particles to bind to trapping sites, which contain binding agents for the particles such as antigens or antibodies, further along the strip. Accumulation of the coloured particles at these sites results in colour development that are dependent on the concentration of competing antigen in the sample. Other dipstick methods may employ antibodies covalently bound to paper or membrane strips to trap antigen in the sample. Subsequent reactions employing second antibodies coupled to enzymes such as horse radish peroxidase and incubation with substrates to produce colour, fluorescent or chemiluminescent light output will enable quantitation of antigen in the sample.

A radioimmunoassay (RIA) may also be used. In one RIA a radiolabelled antigen and unlabelled antigen are employed in competitive binding with an antibody. Common radiolabels include ¹²⁵I, ¹³¹I, ³H and ¹⁴C. Radioimmunoassays involving precipitation of BNP, BNPsp or fragments thereof with a specific antibody and radiolabelled antibody binding protein can measure the amount of labelled antibody in the precipitate as proportional to the amount of the BNP or BNPsp in the sample. Alternatively, a labelled BNP, BNPsp or fragment thereof is produced and an unlabelled antibody binding protein is used. A biological sample to be tested is then added. The decrease in counts from the labelled BNP, BNPsp or fragment thereof is proportional to the amount of BNP, BNPsp or fragment thereof in the sample.

In RIA it is also feasible to separate bound BNP, BNPsp or fragment thereof from free BNP, BNPsp or fragment thereof. This may involve precipitating the BNP/antibody or BNPsp/antibody complex with a second antibody. For example, if the BNP/antibody or BNPsp/antibody complex contains rabbit antibody then donkey anti-rabbit antibody can be used to precipitate the complex and the amount of label counted. For example in an LKB, Gammamaster counter [83].

### Receiver Operating Characteristic (ROC) Analysis

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, a receiver operating characteristic (ROC) plot depicts the overlap between the two distributions by plotting the sensitivity versus 1−specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1−specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test.

One convenient objective to quantify the diagnostic accuracy of a laboratory test is to express its performance by a single number. The most common global measure is the area under the ROC plot. By convention, this area is always ≥0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

### Kits & Articles of Manufacture

The present invention also relates to devices and kits for performing the assays and methods described herein. Suitable kits comprise reagents sufficient for performing an assay for at least one of the described BNP or BNPsp species, together with instructions for performing the described threshold comparisons. For example, kits will be formatted for assays known in the art, and in particular, ELISA assays.

In one aspect of the present invention there is a kit or article of manufacture comprising:
(i) reagents specific to measure the levels of a Type-B natriuretic peptide signal peptide (BNPsp) fragment, the level of N-terminal Type-B natriuretic peptide (NT-proBNP) and white blood cell count (WCC) in a biological sample obtained from a patient; and
(ii) instructions for how to perform the diagnosis of unstable angina in the patient or for how to predict a complication of stroke and/or heart failure as a consequence of developing unstable angina in the patient.

In certain examples, reagents for performing such assays are provided in an assay device, and such assay devices may be included in such a kit. For example, preferred reagents can comprise one or more solid phase antibodies, the solid phase antibody comprising an antibody that detects the BNP or BNPsp species bound to a solid support.

Accordingly, in certain examples of the present invention, the first binding agent is immobilised on a solid support.

In the case of sandwich immunoassays, such reagents can also include one or more detectably labelled antibodies, the detectably labelled antibody comprising an antibody that detects the intended BNP or BNPsp species bound to a detectable label. Additional optional elements that may be provided as part of an assay device are described hereinafter. Detectable labels may include molecules that are themselves detectable (e.g., fluorescent moieties, electrochemical labels, electrochemical luminescence (ecl) labels, metal chelates, colloidal metal particles, etc.) as well as molecules that may be indirectly detected by production of a detectable reaction product (e.g., enzymes such as horseradish peroxidase, alkaline phosphatase, etc.) or through the use of a specific binding molecule which itself may be detectable (e.g., a labelled antibody that binds to the second antibody, biotin, digoxigenin, maltose, oligohistidine, 2,4-dintrobenzene, phenylarsenate, ssDNA, dsDNA, etc.).

As such, in other examples of the present invention, the second binding agent comprises a detectable label.

As described herein, the binding agents comprised within the kits of the present invention may include an antibody or an antigen binding fragment thereof, for example, a monoclonal antibody or antigen binding fragment thereof. A detailed description with respect to binding members, including antibodies and antigen binding fragments is described elsewhere herein.

In certain aspects, the kit comprises reagents for the analysis of at least one test sample. The kit can also include devices and instructions for performing one or more of the diagnostic and/or prognostic correlations described herein. Preferred kits will comprise an antibody pair for performing a sandwich assay, or a labelled species for performing a competitive assay, for an analyte, such as BNP or BNPsp. Preferably, an antibody pair comprises a first antibody conjugated to a solid phase and a second antibody conjugated to a detectable label, wherein each of the first and second antibodies will bind different forms of BNP or BNPsp. Typically, and for the sake of specificity, each of the antibodies used in the kits of the present invention include monoclonal antibodies. The instructions for use of the kit and performing the correlations can be in the form of labelling, which refers to any written or recorded material that is attached to, or otherwise accompanies a kit at any time during its manufacture, transport, sale or use. For example, the term labelling encompasses advertising leaflets and brochures, packaging materials, instructions, audio or video cassettes, computer discs, as well as writing imprinted directly on kits.

Further encompassed within the scope of the present invention are kits comprising dual purpose or multi-site assays for the detection and measurement of different species of BNP including BNP and BNPsp. That is, the present invention provides assays and kits capable of simultaneously determining the presence and amount of different species of BNP and BNPsp, in a biological sample that has been obtained from a subject. In certain examples, the present invention provides dual purpose assays and kits comprising dual purpose assays for the simultaneous measurement of BNP and BNPsp, as well as fragments thereof, wherein the assay comprises any combination of the assays described herein.

Any reference to prior art documents in this specification is not to be considered an admission that such prior art is widely known or forms part of the common general knowledge in the field.

The invention is further described with reference to the following examples. It will be appreciated that the invention as claimed is not intended to be limited in any way by these examples.

### EXAMPLES

### Example 1: Materials and Methods

### Study Population and Design

Patients with chest pain suspicious of acute coronary syndromes (ACS) were prospectively enrolled into our ongoing observational study known as Signal Peptides in Acute Coronary Events (SPACE, http://www.anzctr.org.au, ACTRN12609000057280). All patients were enrolled in accord with protocols approved by the Health and Disabilities Ethics Committee of the Ministry of Health, New Zealand. All participants gave informed consent before recruitment and all investigations conformed to the principles of the Declaration of Helsinki. Between March 2009 and September 2013, 505 eligible patients aged 18 years or older with the primary complaint of acute chest pain clinically suspicious of ACS and ≤4 hours from onset were recruited. More general/atypical symptoms (such as fatigue, nausea, vomiting, sweating and faintness) were not used as inclusion criteria. Patients with end stage renal disease on dialysis were excluded.

### Adjudicated Diagnosis

The adjudicated diagnosis of acute MI was made in accordance with published guidelines [1], by two independent cardiologists with access to all clinical data, but not BNPsp or hsTnT results. In the case of disagreement, an independent third cardiologist adjudicated to resolve this. The biochemical component of the diagnosis of MI was based on a contemporary TnI assay (not highly sensitive) with 1 value ≥99th URL (99th percentile=0.03 μg/L) within 12 hours of presentation. Atrial fibrillation (AF) during emergency department presentation was determined from the ECG, whereas the diagnosis of UAP was made on the basis of confirmatory provocative investigations (exercise tolerance testing (EU) or dobutamine stress echocardiography testing (DSE)) or angiographic catheterisation findings.

### Follow Up and Prognostic End Points

Within 365 days post-discharge, patients were followed up by telephone or in writing. Reported clinical events were identified from the patients themselves (or their primary physician) corroborated by the records of the treating institution or by the centralised New Zealand Ministry of Health database registry entries on mortality and events. The post-discharge end points considered were death, MI, acute decompensated heart failure and stroke. Events were analysed by ROC analysis for three groups; all patients (n=505), MI patients (n=115) and non-MI patients (n=390).

### Clinical Assessment and Sample Collection

For all patients, initial assessment included clinical history, physical examination, ECG recordings, standard blood tests, pulse oximetry and chest radiography. Patient management was at the discretion of the attending physicians. Only standard clinical core lab TnI (Abbott Architect, non-high sensitive index test available at time of study initiation) and other standard blood test results were available to treating staff.

After consent was given, serial blood samples for measurement of BNPsp, NT-proBNP and hsTnT (EDTA tubes) and TnI and lipids (Heparin tubes) were taken at 0, 1, 2 and 12-24 hours after presentation. Blood samples (10 ml) were drawn into EDTA tubes chilled on ice, centrifuged at 2500 g for 10 minutes and the plasma frozen at −80° C. prior to assays. Heparin samples were collected into 5 ml tubes and immediately sent to the hospital core biochemistry unit for measurement of cTnI and lipids.

### BNPsp Assay

BNPsp was measured using our previously reported assay [7-10]. Briefly, the assay has a sample detection limit of 5.0±0.6 pmol/L, ED₅₀ of 161±8 pmol/L and a sample working range of 4-112 pmol/L in which the intra-assay CV is <10%. Inter-assay CVs are ˜14% at 130 pmol/L and ˜13% at 44 pmol/L respectively. The 99^{th} percentile upper limit of the normal range for BNPsp is 25 pmol/L at which the intra-assay CV is 6.2%. Cross-reactivity assessment shows no detectable interference with other relevant peptides or with medications commonly used in cardiovascular disorders.

### Cardiac and Other Marker Assays

NT-proBNP and hsTnT were determined on a Cobas e411 analyser (Roche Diagnostics). The limit of detection (LOD) for the NT-proBNP assay was 5 ng/L and had an imprecision co-efficient of variation (CV) of 4.6% at 44 ng/L. The LOD for the hsTnT assay was 5 ng/L with an imprecision CV of <10% at 13 ng/L. For the purposes of this study, an hsTnT value of 14 ng/L was used as the upper limit of normal cut-off and the clinical threshold for the diagnosis of MI [11]. All hsTnT results were submitted to Penzberg during the worldwide reassessment of hsTnT by Roche and only 3 required adjustment, all of which were below 14 ng/L. TnI was determined by a contemporary assay (Abbott Architect) with a 99th percentile cut-off of 30 ng/L (0.03 ug/L). Cholesterol, HDL, LDL and Triglycerides were determined by the core Christchurch hospital lab (Canterbury Health Laboratories) on an Abbott Series C analyser.

### Statistical Analysis

Continuous variables are presented as median (interquartile range, (IQR)) and categorical variables as numbers and percentages. Bivariate associations between patient outcomes and continuous variables were analysed using non-parametric Mann-Whitney U test and categorical variables using the Pearson χ2 test. Analysis of plasma analyte results employed Spearman rank order correlation testing and receiver operator characteristic curve (ROC) analysis and diagnostic performance (sensitivity, specificity, positive predictive value (PPV) and negative predictive values (NPV)) were carried out using SPSS v22 (IBM). For ROC curve generation and biomarker panel comparisons, biomarker data were analysed as standardised variables (z-scores). In all cases, the standardised variable was derived from the maximum biomarker value obtained from the t=0, 1 and 2 hour samples.

Individual biomarkers (BNPsp, NT-proBNP, TnI and hsTnT) were assessed by ROC analysis for the prediction of index MI and UAP. Combinatorial assessment of standardised biomarkers for the detection of index UAP, thus generating a ratio here termed “UARatio”, was made using analytes according to whether ROC analysis indicated a lower or higher value. Thus, the UARatio exploits lower ROC values which have increased separation from higher ROC values, compared with neutral performers (˜0.5), to predict index UAP. Higher ROC analytes function as numerators, whereas lower ROC values function as denominators. Iterative analysis identified a minimum core set of best performing standardised markers, whose additive nature was confirmed by singular removal and addition, whilst consistency was assessed in 3 randomly selected study population halves.

ROC curve comparisons were made using the approach of Hanley and McNeill [12]. In all analyses, a p-value <0.05 was considered significant.

### Example 2: Results

### Characteristics of Patients

The baseline characteristics for the 505 patients recruited are shown in Table 1. One hundred fifteen (23%) had an adjudicated diagnosis of MI, 40 (8%) had UAP, 324 (64%) had undifferentiated or non-cardiac chest pain and 26 (5%) had a non-ACS cardiac disorder such as atrial fibrillation (AF), heart failure or aortic stenosis. Of these alternate cardiac disorders, 19 (4%) were in AF during their emergency department presentation.

### Biomarker Levels

Presentation BNPsp levels were inversely associated with height (r=−0.13, p=0.006) and positively associated with WCC (r=0.17, P<0.001), HDL (r=0.10, p=0.035), NT-proBNP (r=0.10, p=0.043), TnI (r=0.11, p=0.01) and hsTnT (r=0.10, p=0.029). Plasma BNPsp was significantly higher in MI and other cardiac disorder groups, compared with other diagnoses (Table 1). With the MI cases removed, BNPsp levels were significantly higher in UAP patients, compared with other diagnoses (Table I). Interestingly, presentation levels of BNPsp were significantly higher (p=0.018) in those in AF (25.9 (19.8-36.0 pmol/L), n=19) vs those without AF (22.2 (18.3-25.9 pmol/L), n=486). Both hsTnT and TnI were significantly elevated in MI (as expected) and hsTnT was also significantly elevated in other cardiac disorders (Table I). NT-proBNP levels were elevated in all cardiac disorder groups compared with non-cardiac cases or those with undifferentiated chest pain.

### Diagnosis of MI

The index TnI assay had an ROC AUC of 0.97 for the diagnosis of MI, whereas the investigational hsTnT measurement generated an AUC of 0.96 (Table II, FIG. 3). Both assays generated high sensitivity, specificity, PPV and NPV data. In contrast, BNPsp generated an AUC=0.69 and NT-proBNP=0.64. Addition of BNPsp to TnI, hsTnT or NT-proBNP did not improve their respective AUC's, sensitivity or specificity.

### Identification of Cardiac Ischemia and Other Diagnoses

In the whole study group, no marker AUC fell beyond the line of non-discrimination for the detection of UAP, the closest being NT-proBNP with an AUC of 0.58 (95% CI, 0.50-0.67, p=0.079). When patients with MI (n=115) were removed, this resulted in 390 patients eligible for analysis. ROC analysis on this group revealed that only proBNP, BNPsp, potassium and white cell count (WCC) generated significant AUC's for the identification of patients with adjudicated UAP (Table II). The UARatio generated an AUC of 0.70 for the identification of patients with UAP (Table II) which was significantly better than the best individual marker, NT-proBNP (p<0.05, FIG. 4A). Accordingly, median standardised values of the UARatio were significantly higher in patients with adjudicated UAP compared with all other diagnoses (1.19 (0.49-2.92) vs. 0.43 (0.24-1.29), p=0.002). Further analysis, focussed on non-MI individuals with no evidence of ECG abnormalities (n=328), revealed that the UARatio had an AUC for the identification of UAP of 0.76 (n=33, 95% CI, 0.68-0.83, p<0.001, FIG. 4B) with sensitivity, specificity, PPV and NPV of 90%, 50%, 18% and 98%, respectively. This AUC was significantly better (p<0.05) than the comparative AUC's for NT-proBNP (AUC=0.66, 95% CI 0.57-0.75) and BNPsp (AUC=0.63, 95% CI, 0.52-0.74, FIG. 4B). Serial singular removal of individual variables from the UARatio and repeated assessment of its performance in 3 randomly selected halves of the study population confirmed the consistent test performance of the ratio.

TABLE 1
Baseline characteristics of patients (median, IQR, percent)

| | | | Unstable | | | | | | | | |
|---|---|---|---|---|---|---|---|---|---|---|---|
| | | | angina | | | | Undifferentiated/ | | | | |
| | Myocardial | | pectoris | | Other cardiac | | Non-cardiac | | | | |
| | infarction (MI) | | (UAP) | | disorder | | chest pain | | All patients | | p-value |
| Patient, no. (%) | 115 | (23) | 40 | (8) | 26 | (5) | 324 | (64) | 505 | (100) | |
| Gender, no. (%) | | | | | | | | | | | |
| Male | 78 | (68) | 26 | (65) | 16 | (62) | 185 | (57) | 305 | (60) | <0.001 |
| Female | 37 | (32) | 14 | (35) | 10 | (38) | 139 | (43) | 200 | (40) | <0.001 |
| Age, yrs | | | | | | | | | | | |
| Male | 66 | (56-76) | 64 | (58-70) | 65 | (52-77) | 59 | (48-70) | 62 | (51-70) | <0.001 |
| Female | 78 | (68-86) | 66 | (59-73) | 73 | (65-80) | 68 | (58-80) | 68 | (59-80) | <0.001 |
| Analytes | | | | | | | | | | | |
| Chol (mg · dL⁻¹)ψ | 181 | (154-216) | 175 | (141-213) | 174 | (149-202) | 187 | (153-214) | 184 | (154-216) | |
| HDL (mg · dL⁻¹)ψ | 40 | (34-50) | 39 | (35-42) | 40 | (30-58) | 40 | (37-53) | 40 | (36-51) | |
| LDL (mg · dL⁻¹)ψ | 116 | (93-143) | 107 | (77-139) | 101 | (85-125) | 116 | (89-135) | 114 | (89-135) | |
| Trig (mg · dL⁻¹) § | 159 | (97-195) | 149 | (95-177) | 142 | (88-181) | 166 | (126-186) | 161 | (107-206) | |
| Risk factor (%) | | | | | | | | | | | |
| Hypertension | 79 | (69) | 33 | (83) | 20 | (77) | 195 | (60) | 327 | (65) | <0.01 |
| Diabetes | 19 | (4) | 9 | (23) | 4 | (15) | 43 | (13) | 75 | (15) | <0.01 |
| Current smoker | 16 | (3.5) | 2 | (5) | 0 | (0) | 47 | (15) | 65 | (13) | <0.01 |
| Ever smoker | 57 | (11) | 21 | (53) | 19 | (73) | 157 | (48) | 254 | (50) | <0.01 |
| BMI (kg · m²) | 27.7 | (24.7-31.3) | 27.2 | (25.1-30.3) | 27.7 | (24.6-31.8) | 27.7 | (24.9-31.3) | 27.7 | (24.7-31.1) | |
| History (%) | | | | | | | | | | | |
| CVD | 78 | (68) | 36 | (90) | 13 | (50) | 199 | (61) | 326 | (65) | <0.01 |
| MI | 36 | (31) | 20 | (50) | 10 | (38) | 99 | (31) | 165 | (33) | <0.01 |
| CABG | 9 | (8) | 6 | (15) | 3 | (12) | 35 | (11) | 53 | (10) | <0.01 |
| Hyperlipidaemia | 62 | (54) | 34 | (85) | 14 | (54) | 193 | (60) | 303 | (60) | <0.01 |
| Angina | 48 | (42) | 30 | (75) | 17 | (65) | 158 | (49) | 253 | (50) | <0.01 |
| Heart failure | 10 | (9) | 4 | (10) | 2 | (8) | 33 | (10) | 49 | (10) | <0.01 |
| ECG Results (%) | | | | | | | | | | | |
| LBBB | 3 | (3) | 1 | (3) | 1 | (4) | 9 | (3) | 14 | (4) | |
| ST-elevation | 23 | (20) | 0 | (0) | 2 | (8) | 0 | (0) | 25 | (7) | <0.01 |
| ST-depression | 10 | (9) | 1 | (3) | 3 | (12) | 2 | (1) | 16 | (4) | <0.01 |
| T-wave inversion | 20 | (17) | 5 | (13) | 6 | (23) | 30 | (9) | 61 | (13) | <0.01 |
| No change | 59 | (51) | 33 | (83) | 14 | (54) | 283 | (9) | 389 | (72) | <0.01 |
| Marker Levels | | | | | | | | | | | |
| hsTnT(ng/L) | 79* | (37-219) | 6 | (3-10) | 22* | (8-36) | 5 | (3-12) | 8 | (3-27) | *<0.01 |
| TnI (μg/L) | 0.25* | (0.07-1.30) | 0.01 | (0.01-0.01) | 0.02 | (0.01-0.04) | 0.01 | (0.01-0.01) | 0.01 | (0.01-0.03) | *<0.01 |
| BNPsp (pmol/L) | 26.1* | (20.5-36.9) | 23.1^{†} | (18.1-28.2) | 25.0* | (19.6-34.0) | 21.3 | (17.7-24.2) | 22.3 | (18.4-26.2) | *<0.01 |
| | | | | | | | | | | | ^{†}<0.05 |
| proBNP(ng/L) | 758* | (279-1447) | 436* | (288-1445) | 1159* | (445-2013) | 340 | (157-767) | 392 | (183-1063) | *<0.01 |
| UARatio | 0.44* | (−0.81-1.96) | 0.23* | (−0.50-1.94) | 0.01 | (−0.51-1.40) | −0.76 | (−1.37-0.16) | −0.35 | (−1.09-0.76) | *<0.01 |

TABLE II
ROC curve parameters for diagnosis of MI and definite unstable angina (UAP)

| | | | | | | | |
|---|---|---|---|---|---|---|---|
| Marker | AUC | 95% CI | Sens. % | Spec. % | PPV % | NPV % | p-value |

Diagnosis of MI (n = 115/505)

| | | | | | | | |
|---|---|---|---|---|---|---|---|
| TnI | 0.97 | 0.96-0.99 | 85 | 95 | 83 | 96 | <0.001 |
| hsTnT | 0.96 | 0.94-0.98 | 94 | 83 | 62 | 98 | <0.001 |
| NT-proBNP | 0.64 | 0.58-0.69 | 56 | 73 | 38 | 85 | <0.001 |
| BNPsp | 0.69 | 0.63-0.75 | 55 | 84 | 50 | 86 | <0.001 |
| UARatio | 0.64 | 0.58-0.70 | 60 | 68 | 36 | 85 | <0.001 |

Diagnosis of UAP (n = 40/390)

| | | | | | | | |
|---|---|---|---|---|---|---|---|
| TnI | 0.54 | 0.44-0.64 | 5 | 85 | 4 | 89 | 0.419 |
| hsTnT | 0.51 | 0.42-0.59 | 10 | 80 | 5 | 89 | 0.910 |
| NT-proBNP | 0.62 | 0.54-0.70 | 83 | 37 | 13 | 95 | 0.012 |
| Potassium | 0.61 | 0.53-0.70 | 5 | 97 | 16 | 90 | 0.019 |
| WCC¹ | 0.60 | 0.52-0.69 | 5 | 93 | 8 | 90 | 0.032 |
| BNPsp | 0.59 | 0.49-0.70 | 83 | 45 | 15 | 96 | 0.048 |
| UARatio² | 0.70* | 0.62-0.77 | 83 | 51 | 16 | 97 | <0.001 |

TABLE III
ROC performance (AUC, 95% CI) of presentation marker
levels to identify outcomes within 1 year.
Whole study group (n = 505)

| | Mortality | MI | Stroke | Heart failure |
|---|---|---|---|---|
| Marker | (n = 21) | (n = 29) | (n = 10) | (n = 10) |
| TnI | 0.70* | 0.64* | 0.51 | 0.72† |
| | (0.59-0.80) | (0.54-0.74) | (0.34-0.68) | (0.58-0.86) |
| hsTnT | 0.70* | 0.68* | 0.68 | 0.82* |
| | (0.60-0.80) | (0.59-0.77) | (0.55-0.80) | (0.73-0.90) |
| NT-proBNP | 0.73* | 0.70* | 0.77* | 0.92* |
| | (0.63-0.74) | (0.60-0.79) | (0.64-0.90) | (0.84-1.00) |
| BNPsp | 0.59 | 0.44 | 0.48 | 0.51 |
| | (0.49-0.69) | (0.33-0.55) | (0.31-0.64) | (0.34-0.69) |
| UARatio | 0.61 | 0.62† | 0.63 | 0.81* |
| | (0.48-0.75) | (0.52-0.72) | (0.49-0.77) | (0.70-0.92) |

MI patients (n = 115)

| | Mortality | MI | | |
|---|---|---|---|---|
| Marker | (n = 7) | (n = 13) | | |
| NT-proBNP | 0.71 | 0.63 | nd | nd |
| | (0.48-0.94) | (0.47-0.78) | | |
| BNPsp | 0.54 | 0.71† | nd | nd |
| | (0.49-0.69) | (0.57-0.85) | | |
| NT-proBNP + | 0.67 | 0.70† | nd | nd |
| BNPsp | (0.40-0.93) | (0.56-0.84) | | |

Non-MI patients (n = 390)

| | Mortality | MI | Stroke | Heart failure |
|---|---|---|---|---|
| Marker | (n = 14) | (n = 16) | (n = 9) | (n = 7) |
| UARatio | 0.62 | 0.61 | 0.70† | 0.82* |
| | (0.48-0.77) | (0.48-0.74) | (0.55-0.85) | (0.67-0.96) |
| BNPsp | 0.59 | 0.47 | 0.50 | 0.61 |
| | (0.48-0.70) | (0.33-0.62) | (0.31-0.65) | (0.37-0.85) |

†= p < 0.05,
*= p < 0.01.
nd = not determined

### Prognostic Ability of BNPsp and UARatio

Assessment of the whole study group (n=505) for the prognostic performance of BNPsp to 1 year from index admission revealed that BNPsp at presentation did not predict mortality, myocardial infarction, stroke or heart failure. Furthermore, BNPsp did not add to the predictive abilities of hsTnT, TnI or NT-proBNP with respect to those outcomes (Table III). In contrast, the variable UARatio predicted MI (n=29, p=0.029) and heart failure (n=10, p=0.001) within one year (Table III).

Focus on MI patients alone (n=115) revealed BNPsp concentrations generated an AUC of 0.71 (p=0.014) with BNPsp <26 pmol/L significantly associated with new MI (n=13) within one year. Adding BNPsp improved the AUC of NT-proBNP for prediction of new MI within one year from 0.63 (p=0.136) to 0.70 (p=0.021, Table III). UARatio did not predict any outcomes in MI patients. Analysis of individuals who did not suffer MI (n=390) revealed that the UARatio could predict stroke (n=9, P=0.038) and heart failure (n=7, p=0.004) within one year (Table III). BNPsp did not predict any events in non-MI patients.

### Example 3: Discussion

Applicant's earlier work in identifying BNPsp as a circulating entity with a rapidly rising profile in both acute MI [7], and on provocative cardiac testing [10], provided the rationale for the study presented here. The major findings of this work are: i) BNPsp had similar ROC diagnostic power for acute MI to copeptin (˜0.7) in ED patients with chest pain [13]. However, unlike copeptin, it did not add diagnostic power to troponin; ii) BNPsp levels are significantly elevated in patients presenting with AF, iii) BNPsp, along with NT-proBNP, had some discriminative power for UAP in non-MI individuals and the additive value of these two combined with WCC and K+ gave rise to a unique ratio that may have diagnostic potential in UAP, especially in patients with no change on their ECG, and iv) BNPsp might add to the prognostic information from NT-proBNP in patients suffering acute MI.

The finding that BNPsp levels within two hours of presentation were elevated in acute MI is consistent with our previous findings in STEMI patients [7]. Furthermore, this study has also confirmed the highly dynamic nature of BNPsp elevations in cardiac ischemia, in that elevations in this study were rapid in their onset and offset. This pattern of BNPsp release might make it useful in terms of detecting repeated ischemic episodes, but renders elevations more difficult to detect and requires repeated sampling. Rapid half-life and clearance from the circulation is likely one reason why BNPsp did not add to hsTnT measurement for the diagnosis of MI.

Elevations of BNPsp in patients with AF is a novel finding. The underlying mechanism is unknown but could reflect rapidly changing local mechanical stresses upon varying populations of atrial myocytes and/or tachycardia induced ischemia. The relationship of BNP/NT-proBNP with AF occurrence and risk prediction is well known [14-16], as is that of troponin [17,18], and it would be of interest to determine if BNPsp has similar capabilities in a larger, appropriately designed study sample.

The potential ability of BNP to detect cardiac ischemia short of infarction has evidential backing from experimental [19], clinical [20], and meta-analysis study [21], which all suggest that BNP measurement can improve detection of myocardial ischemia during provocative testing regimes. Our data that BNPsp was somewhat discriminative of UAP in non-MI patients, albeit not as strongly as NT-proBNP, is a positive finding and whilst consistent with our previous report in CAD patients undergoing stress echocardiography testing [10], was generated from individuals who did not receive any test stimulus or provocation. The addition of BNPsp to NT-proBNP was further developed into the concept of a “UARatio” in which other variables displaying significant ROC responses in UAP patients were also included. The rationale behind the UARatio is an attempt to include multiple, potentially useful biomarker values that differentially respond to the syndrome of interest. Of the four variables identified here, BNPsp and NT-proBNP are intuitively appropriate, the other two less so. The use of WCC on the denominator reflects that fact that in this study, WCC levels were significantly lower (P=0.01) in UAP compared with all other diagnostic groups. In contrast, WCC values were significantly higher (P<0.01) in acute MI patients. There is variation in the literature with respect to WCC levels in UAP patients with reports they are elevated [22], unchanged [23], and decreased [24]. This variation is likely to reflect time of sampling, prior medication history and smoking status of the study groups. In contrast, we found elevations (non-significant) in potassium levels in UAP patients, which generated a weak, but significant ROC AUC and it is noteworthy that potassium combined positively with each of, and the combination of, NT-proBNP and BNPsp in the detection of UAP.

With respect to the UARatio suggested here, the PPV and NPV values generated in UAP patients, especially in the equivocal ECG group, are within the ranges reported for exercise and echocardiographic testing combined [25]. Furthermore, the UARatio had prognostic ability for subsequent MI, stroke and episode of heart failure within one year. Future studies might address whether; 1) the ratio described here can improve the low diagnostic and therapeutic yields from current provocative cardiac testing regimes [26], and 2) there is any combining the ratio with other novel potential markers of UAP such as microRNAs [27].

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Thygesen K, Alpert J S, Jaffe A S, Simoons M L, Chaitman B R, White H D; Joint ESC/ACCF/AHA/WHF Task Force for the Universal Definition of Myocardial Infarction. Third universal definition of myocardial infarction. Circulation 2012; 126:2020-35.
2. Keller T, Zeller T, Peetz D, Tzikas S, Roth A, Czyz E, et al. Sensitive troponin I assay in early diagnosis of acute myocardial infarction. N Engl J Med 2009; 361:868-77.
3. Reichlin T, Hochholzer W, Bassetti S, Steuer S, Stelzig C, Hartwiger S, et al. Early diagnosis of myocardial infarction with sensitive cardiac troponin assays. N Engl J Med 2009; 361:858-67.
4. Reichlin T, Twerenbold R, Reiter M, Steuer S, Bassetti S, Balmelli C, et al. Introduction of high sensitivity troponin assays: impact on myocardial infarction incidence and prognosis. Am J Med 2012; 125:1205-13.
5. Thygesen K, Mair J, Giannitsis E, Mueller C, Lindahl B, Blankenberg S, et al. Study Group on Biomarkers in Cardiology of ESC Working Group on Acute Cardiac Care. How to use high-sensitivity cardiac troponins in acute cardiac care. Eur Heart J 2012; 33:2252-57.
6. Hamm C W, Bassand J P, Agewall S, Bax J, Boersma E, Bueno H, et al. ESC Committee for Practice Guidelines. ESC Guidelines for the management of acute coronary syndromes in patients presenting without persistent ST-segment elevation: The Task Force for the management of acute coronary syndromes (ACS) in patients presenting without persistent ST-segment elevation of the European Society of Cardiology (ESC). Eur Heart J 2011; 32:2999-3054.
7. Siriwardena M, Kleffmann T, Ruygrok P, Cameron V A, Yandle T G, Nicholls M G, et al. BNP signal peptide circulates in human blood: evaluation as a potential biomarker of cardiac ischemia. Circulation 2010; 122:255-64.
8. Pemberton C J, Siriwardena M, Kleffmann T, Ruygrok P, Palmer S C, Yandle T G, et al. First identification of circulating preproANP signal peptide fragments in man: initial assessment as cardiovascular biomarkers. Clin Chem 2012; 58:757-77.
9. Pemberton C J, Siriwardena M, Kleffmann T, Richards A M. CNP signal peptide fragments are present in the human circulation. Biochem Biophys Res Commun 2014; 449:301-306.
10. Siriwardena M, Campbell V, Richards A M, Pemberton C J. Cardiac biomarker responses to dobutamine stress echocardiography in healthy volunteers and patients with coronary artery disease. Clin Chem 2012; 58:1492-94.
11. Giannitsis E, Kurz K, Hallermayer K, Jarausch J, Jaffe A S, Katus H A. Analytical validation of a high-sensitivity cardiac troponin T assay. Clin Chem 2010; 56:254-61.
12. Hanley J A, McNeill B J. A method of comparing the areas under receiver operating characteristic curves derived from the same cases. Radiology 1983; 148:839-43.
13. Raskovalova T, Twerenbold R, Collinson P O, Keller T, Bouvaist H, Folli C, et al. Diagnostic accuracy of combined cardiac troponin and copeptin assessment for early rule-out of myocardial infarction: a systematic review and meta-analysis. Eur Heart J Acute Cardiovasc Care 2014; 3:18-27.
14. Patton K K, Ellinor P T, Heckbert S, Christenson R H, DeFilippi C, Gottdiener J S, et al. N-terminal pro-B-type natriuretic peptide is a major predictor of the development of atrial fibrillation: the Cardiovascular Health Study. Circulation 2009; 120:1768-74.
15. Schnabel R B, Larson M G, Yamamoto J F, Sullivan L M, Pencina M J, Meigs J B, et al. Relations of biomarkers of distinct pathophysiological pathways and atrial fibrillation incidence in the community. Circulation 2010; 121:200-207.
16. Sinner M F, Stepas K A, Moser C B, Krijthe B P, Aspelund T, Sotoodehnia N, et al. B-type natriuretic peptide and C-reactive protein in the prediction of atrial fibrillation risk: the CHARGE-AF consortium of community based cohort studies. Europace 2014; 16:1426-33.
17. Hussein A A, Bartz T M, Gottdiener J S, Sotoodehnia N, Heckbert S R, Lloyd-Jones D, et al. Serial measures of cardiac troponin T levels by a highly sensitive assay and incident atrial fibrillation in a prospective cohort of older ambulatory adults. Heart Rhythm 2015: http//:dx.doi.org/10.1016.j.hrthm.2015.01.020
18. Filion K B, Agarwal S K, Ballantyne C M, Eberg M, Hoogeveen R C, Huxley R R, et al. High sensitivity cardiac troponin T and the risk of incident atrial fibrillation: the Atherosclerosis Risk in Communities (ARIC) Study. Am Heart J 2015; 169:31-8.
19. Mollmann H, Nef H M, Kostin S, Dragu A, Maack C, Weber M, et al. Ischemia triggers BNP expression in the human myocardium independent from mechanical stress. Int J Cardiol 2010; 141; 265-71.
20. Staub D, Jonas N, Zellweger M J, Nusbaumer C, Wild D, Pfisterer M E, et al. Use of N-terminal pro-B-type natriuretic peptide to detect myocardial ischemia. Am J Med 2005; 118:1287.
21. Nadir M A, Witham M D, Szwejkowski B R, Struthers A D. Meta-analysis of B-type natriuretic peptide's ability to identify stress induced myocardial ischemia. Am J Cardiol 2011; 107:662-67.
22. Yip H K, Wu C J, Hang C L, Chang H W, Yang C H, Hsieh Y K, et al. Levels and values of inflammatory markers in patients with angina pectoris. Int Heart J 2005; 46:571-81.
23. Avramakis G, Papadimitraki E, Papakonstandinou D, Liakou K, Zidianakis M, Dermitzakis A, et al. Platelets and white blood cell subpopulations among patients with myocardial infarction and unstable angina. Platelets 2007; 18:16-23.
24. Yang N, Feng J P, Chen G, Kou L, Li Y, Ren P, et al. Variability in lipid profile among patients presented with acute myocardial infarction, unstable angina and stable angina pectoris. Eur Rev Med Pharmcol Sci 2014; 18:3761-66.
25. Cheitlin M D, Alpert J S, Armstrong W F, Aurigemma G P, Beller G A, Bierman F Z, et al. ACC/AHA Guidelines for the Clinical Application of Echocardiography. A report of the American College of Cardiology/American Heart Association Task Force on Practice Guidelines (Committee on Clinical Application of Echocardiography). Developed in collaboration with the American Society of Echocardiography. Circulation 1997; 95:1686-1744.
26. Hermann L K, Newman D H, Pleasant A, Rojanasarntikul D, Lakoff D, Goldberg S A, et al. Yield of routine provocative cardiac testing among patients in an emergency department based-chest pain unit. JAMA Intern Med 2013; 173:1128-33.
27. Zeller T, Keller T, Ojeda F, Reichlin T, Twerenbold R, Tzikas S, et al. Assessment of microRNAs in patients with unstable angina pectoris. Eur Heart J 2014; 35:2106-14.
28. Pemberton C J, Johnson M L, Yandle T G, Espiner E A. Deconvolution analysis of cardiac natriuretic peptides during acute volume overload. Hypertension 2000; 36:355-59.
29. Zimmermann R, Baki S, Dengler T J, Ring G H, Remppis A, Lange R, et al. Troponin T release after heart transplantation. Br Heart J 1993; 69:395-98.
30. Dastjerdi et al. 2011 Biotechnology and Applied Biochemistry 58(4):226-230
31. Phillips et al., 2008, Anal Chim Acta 621: 101-108
32. Shamah et al., 2008, Acc Chem Res 41: 130-138
33. Ohuchi et al., 2006. Biochimie 88:897-904
34. Fundamental Immunology, 3rd Edition, W. E. Paul, ed., Raven Press, N.Y. (1993)
35. Wilson (1994) J. Immunol. Methods 175:267-273
36. Yarmush (1992) J. Biochem. Biophys. Methods 25:85-97
37. Ward et al., (1989) Nature 341:544-546
38. Proc. Natl. Acad. Sci. USA 81: 6851-6855 (1984)
39. Protein Eng 8(10) 1057-1062 (1995)
40. The Pharmacology of Monoclonal Antibodies, vol. 113, Springer-verlag 1994, Rosenburg and Moore Eds
41. Proc. Natl. Acad. Sci USA 90: 6444-6448 (1993)
42. Nature 321: 522-525 (1986)
43. Nature 332: 323-329 (1988)
44. WO 2005/003154
45. Kohler and Milstein (1975) Nature (5517) 256, 495-497
46. U.S. Pat. No. 4,196,265
47. U.S. Pat. No. 4,816,567
48. Lutz et al. (1988) Exp. Cell. Res. 175:109-124
49. Munson et al. (1980) Anal Biochem 107: 220
50. U.S. Pat. No. 4,816,567
51. U.S. Pat. No. 5,334,708
52. U.S. Pat. No. 5,821,047
53. U.S. Pat. No. 7,476,724
54. U.S. Pat. No. 4,816,567
55. U.S. Pat. No. 5,843,708
56. U.S. Pat. No. 6,020,153
57. Nature 352: 624-628 (1991)
58. Scopes, Protein Purification: Principles and Practice, Springer-Verlag, NY (1982).
59. van Erp et al. (1991) J. Immunoassay 12: 425-43; Nelson and Griswold (1988) Comput. Methods Programs Biomed. 27: 65-8
60. Cwirla et al. (1990) Proc. Natl. Acad. Sci. USA 87, 6378-82
61. Devlin et al. (1990) Science 249, 404-6
62. Scott and Smith (1990) Science 249, 386-88
63. U.S. Pat. No. 5,571,698
64. U.S. Pat. No. 6,057,098
65. United States Patent Application No. 20120225088
66. U.S. Pat. No. 6,143,576
67. U.S. Pat. No. 6,113,855
68. U.S. Pat. No. 6,019,944
69. U.S. Pat. No. 5,985,579
70. U.S. Pat. No. 5,947,124
71. U.S. Pat. No. 5,939,272
72. U.S. Pat. No. 5,922,615
73. U.S. Pat. No. 5,885,527
74. U.S. Pat. No. 5,851,776
75. U.S. Pat. No. 5,824,799
76. U.S. Pat. No. 5,679,526
77. U.S. Pat. No. 5,525,524
78. U.S. Pat. No. 5,480,792
79. The Immunoassay Handbook, David Wild, ed. Stockton Press, New York, 1994
80. U.S. Pat. No. 5,631,171
81. U.S. Pat. No. 5,955,377
82. U.S. Pat. No. 7,541,160
83. Hunt et al. Clin. Endocrinol. 1997 47:287-296

1. A method for diagnosing unstable angina in a patient, the method comprising:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,
wherein, a ratio of BNPsp fragment and NT-proBNP to WCC that deviates from a reference ratio obtained from a control subject is diagnostic that the patient has unstable angina.

2-18. (canceled)

19. A method for predicting a complication of heart failure or stroke in a patient who has previously been diagnosed with unstable angina, the method comprising:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,
wherein, a ratio of BNPsp fragment and NT-proBNP to WCC that deviates from a reference ratio obtained from a control subject is predictive that the patient will develop a complication of heart failure or stroke as a consequence of unstable angina.

20. The method according to claim 1, further comprising measuring the level of potassium (K) in the biological sample.

21. The method according to claim 1, wherein the BNPsp fragment is a fragment defined by residues 17-26 (BNPsp (17-26)).

22. The method according to claim 1, wherein the level of BNPsp fragment or the level of NT-proBNP is determined by immunoassay or mass spectroscopy.

23. The method according to claim 22, wherein the immunoassay is a quantitative immunoassay.

24. The method according to claim 22, wherein the immunoassay is a sandwich assay, a fluoroimmunoassay, an immunofluorometric assay, an immunoradiometric assay, a luminescence assay or a chemiluminescence assay.

25. The method according to claim 22, wherein the immunoassay comprises a BNPsp fragment binding agent or a NT-proBNP binding agent.

26. The method according to claim 25, wherein the binding agent is an antibody or antigen-binding fragment thereof, or the binding agent is an aptamer.

27. The method according to claim 26, wherein the antibody is selected from the group consisting of a monoclonal antibody, a polyclonal antibody, a chimeric antibody, a bispecific antibody, a humanized antibody, and antigen-binding fragments thereof.

28. The method according to claim 25, wherein the binding member, antibody or aptamer is coupled to a solid phase.

29. The method according to claim 1, wherein diagnosis of unstable angina is characterized using Receiver Operating Curve (ROC) analysis.

30. The method according to claim 29, wherein the Receiver Operating Curve (ROC) analysis comprises normalizing the levels of the BNPsp fragment and NT-proBNP to white blood cell count.

31. The method according to claim 29, wherein the diagnosis of unstable angina characterized by Receiver Operating Curve (ROC) analysis further comprises determining the ratio of (BNPsp fragment+NT-proBNP)/WCC.

32. The method according to claim 1, wherein the diagnosis of unstable angina further comprises administering an intervention therapy to reduce, eliminate, mitigate or treat the unstable angina.

33. The method according to claim 1, wherein the biological sample is selected from the group consisting of whole blood, plasma or serum.

34. A method for diagnosing unstable angina in a patient, the method comprising:
(i) measuring the level of a Type-B natriuretic peptide signal peptide (BNPsp) fragment, the level of N-terminal Type-B natriuretic peptide (NT-proBNP) and white blood cell count (WCC) in a biological sample obtained from the patient; and
(ii) comparing the levels of the BNPsp fragment, NT-proBNP and WCC against reference levels obtained from a control subject,
wherein, a ratio of BNPsp fragment and NT-proBNP to WCC that deviates from a reference ratio obtained from a control subject is diagnostic that the patient has unstable angina, and wherein in the event of a positive diagnosis of unstable angina:
(iii) administering an intervention therapy so as to reduce, eliminate, ameliorate or treat unstable angina in the patient.

35. A kit or article of manufacture comprising:
(i) reagents specific to measure the levels of a Type-B natriuretic peptide signal peptide (BNPsp) fragment, the level of N-terminal Type-B natriuretic peptide (NT-proBNP) and white blood cell count (WCC) in a biological sample obtained from a patient; and
(ii) instructions for how to perform the diagnosis of unstable angina in the patient or for how to predict a complication of stroke or heart failure as a consequence of developing unstable angina in the patient.
